(19) United States
(12) Patent Application Publication
(10) Pub. No.: US 2023/0287075 A1
(43) Pub. Date: Sep. 14, 2023

(54) DUAL CYTOKINE FUSION PROTEINS COMPRISING MULTI-SUBUNIT CYTOKINES

(71) Applicant: Deka Biosciences, Inc., Germantown, MD (US)

(72) Inventor: John MUMM, Germantown, MD (US)

(21) Appl. No.: 18/065,504

(22) Filed: Dec. 13, 2022

Related U.S. Application Data
(60) Provisional application No. 63/265,339, filed on Dec. 13, 2021.
(60) Provisional application No. 63/320,750, filed on Mar. 17, 2022.
(60) Provisional application No. 63/328,990, filed on Apr. 8, 2022.

(51) Int. Cl. C07K 14/54; C07K 16/10; C07K 16/28; C07K 16/32; C07K 14/56; A61P 35/00

(52) U.S. Cl. CPC C07K 14/5434; C07K 16/1045; C07K 16/10; C07K 16/2863; C07K 16/32; C07K 14/56; C07K 14/54; C07K 16/2896; A61P 35/00; C07K 2319/30; A61K 2039/505

(57) ABSTRACT

The application relates to a dual cytokine fusion protein composition, pharmaceutical composition, and/or formulation thereof comprising the alpha and beta multi-subunits cytokines, such as IL-12 or IL-27, fused to a single chain variable fragment scaffolding system and a second cytokine, where the second cytokine is linked in the hinge region of the scFv. The application also relates to methods of using the dual cytokine fusion protein composition for treating cancer, inflammatory diseases or disorders, and immune and immune mediated diseases or disorders.

### CROSS-REFERENCE TO RELATED APPLICATION

The present application claims priority to U.S. Provisional Patent Application Ser. No. 63/265,339 filed on Dec. 13, 2021, the content of which is incorporated herein by reference in its entirety.

### REFERENCE TO AN ELECTRONIC SEQUENCE LISTING

The contents of the electronic sequence listing (039451-00082_Sequence-Listing.xml; Size: 76,662 bytes; and Date of Creation: Dec. 13, 2022) are herein incorporated by reference in its entirety.

### FIELD OF INVENTION

The present disclosure relates to the field of biotechnology, and more specifically, to a novel dual cytokine fusion protein comprising Interleukin-12 (“IL-12”) or Interleukin-IL-27 in combination with other inflammatory and immune regulating cytokines, methods of treating inflammatory and immune disease or conditions, and/or methods of treating cancer.

### INTRODUCTION

IL-12 is a 70 kDa heterodimeric cytokine that is the prototypic T helper 1 (Th1) polarizing cytokine (Mossman, 1989; Athie-Morales, 2004). IL-12 exerts potent anti-tumor immunity through activating CD8+ T cells (Henry, 2008; Vacaflores, 2017; Chowdhury, 2011), NK cells (Martinović, 2015; Parihar, 2002; Zhang, 2008), CD4+ T cells (Yoo, 2002; Vacaflores1, 2016), and to a limited degree, monocytes (Coma, 2006). Therefore IL-12 predominantly enhances antigen specific T cell activation, while partially bridging to the innate immune system through moderate stimulation of both NK cells and monocytes.

Interferon-alpha (IFNα-2a) is monomeric Type I interferon that directly induces dendritic cell maturation (Simmons, 2012; Gessani, 2014; Padovan, 2002) and enhances CD8+ T cell cytotoxic function (Hiroishi, 2000; Kolumam, 2005; Lu, 2019). Therefore, IFNα-2a exhibits greater function on the innate immune system compared to it's more limited effects on the adaptive immune response.

The anti-tumor effects of IL-12 have been evaluated preclinically and clinically (Brunda, 1993; Atkins, 1997), where due to toxicity, direct intratumor injection was evaluated and found to be superior to clinical administration (Herpen, 2004; Li S., 2005; Sabel, 2004).

Similarly, IFNα-2a has been evaluated preclinically and in the pegylated form clinically (Lyrdal, 2009; Sunela, 2009; Medrano, 2017), and approved for use in some cancers (How, 2020).

Therefore, given the need for both priming the anti-tumor immune response (Nemunaitis, 2005), and stimulation of CD8+, CD4+ and NK cells to drive robust anti-tumor function, the inventor has found that combining both IFNα-2a and IL-12 on a targeting dual cytokine scaffold system (known as a “Diakine™, (“DK”), which was generally described in co-pending application U.S. application Ser. No. 17/110,104) to target these two cytokines into the tumor microenvironment, enhances in situ priming via inducing the differentiation of M2 monocytes to functional antigen presenting cells (Vidyarthi, 2018), and enhances T and NK cell function. The combination of these two cytokines bridges stimulation between the adaptive and innate immune systems.

Interleukin 10 (IL-10) is a non-covalent homo-dimeric cytokine with structural similarities to Interferon g (IFNg). The IL-10 receptor consists of two molecules of the IL10 receptor 1 (IL10R1) and two molecules of the IL-10 receptor 2 (IL10R2) (Moore, 2001). The IL-10 receptor is expressed on the surface of most hematopoietic cells and is highly expressed on macrophages and T-cells.

While IL-10 has been reported to be both an immunosuppressive (Schreiber, 2000) and immunostimulatory cytokine (Mumm, 2011), clinical evaluation of IL-10 for treating Crohn's patients resulted in an inverse dose response (Fedorak, 2000; Schreiber, 2000), whereas treating cancer patients with PEGylated IL-10 resulted in dose titratable potent anti-tumor responses (Naing, 2018).

IL-10 has been reported to suppress IL-2 driven IFNg production secreted by both NK and CD4+ T cells (Scott, 2006), but it has also been reported to act as a cofactor for IL-2 induced CD8+ T cell proliferation (Groux, 1998).

The inventor has also found that combining both IL-10 and IL-12 on a targeting DK cytokine scaffold system to target these two cytokines into the tumor microenvironment, enhances NK and T cell function.

IL-27 is a member of the IL-12 family and is a heterodimeric cytokine comprised of two subunits, p28 and Epstein Barr virus-induced-gene 3 (“EIB3”). Il-27 is known to induce IL-10. IL-28 is a type 3 interferon that elicits IFNα-2a release stimulating CD8+ T-cells. IL-28 includes two isoforms, IL-28A and IL-28B. IL-29, which shares sequence homology to IL-28, is also a type 3 interferon that is involved in both the innate and adaptive immune response.

The inventor also found that other cytokine combinations including, but not limited to, IL-12 with interleukin-28 (IL-28) or interleukin-29 (IL-29), interleukin-27 (IL-27) with IFNα-2a, IL-28 or IL-29, may be incorporated into the dual cytokine scaffolding system described herein (see, e.g., FIG. 1). Briefly, the dual cytokine scaffolding system permits multi-subunit cytokines (e.g., including but not limited to IL-12, IL-27) to be fused to the terminal ends of a scaffolding system comprising an antigen binding domain or single chain variable region (scFv) of a human anti-HIV or human anti-ebola monoclonal antibody, in combination with a second cytokine (e.g., IL-2, IL-4, IFNa-2a, IL-10), where the second cytokine is fused in the hinge region of the antigen binding domain or scFv.

The inventor also found that the dual cytokine scaffolding system permits multi-subunit cytokines (e.g., IL-12, IL-27) to be fused to the terminal ends of the scaffolding system comprising an antigen binding domain or scFv of a human anti-HIV or human anti-ebola monoclonal antibody, in combination with a monomeric cytokine (e.g., IFN-α, IL-28, IL-29) or another multi-subunit cytokine (e.g., IL-10, IL-12, IL-27).

As previously described in U.S. Pat. No. 10,858,412, a scaffolding system comprising non-immunogenic variable heavy (“VH”) and variable light (“VL”) regions, resulted in the production of a half-life extended IL-10 variant molecules that properly folded and remained functionally active. The incorporation of the IL-10 into the scaffolding system showed enhanced IL-10 function on both inflammatory cells (e.g., monocytes/macrophages/dendritic cells) and immune cells (e.g., CD8⁺ T-cells). See, U.S. Pat. No. 10,858,412; filed on Mar. 6, 2020 as U.S. application Ser. No. 16/811,718, incorporated by reference in its entirety. This application improves on the earlier discovery of an IL-10 based dual cytokine scaffolding system by substituting other multi-subunit based cytokines (e.g., IL-12, IL-27 to name a few) in place of the IL-10 and further incorporating a second cytokine into the new fusion protein that additively or synergistically enhances the biology of the multi-subunit cytokine (e.g., IL-12 or IL-27) to treat inflammatory diseases, immune diseases, and/or cancer.

### SUMMARY OF VARIOUS ASPECTS OF THE INVENTION

The present disclosure generally relates to a dual cytokine fusion protein.

Thus in a first aspect, the present disclosure relates to a dual cytokine fusion protein comprising a first cytokine that is a multi-subunit cytokines, such as but not limited to IL-10, IL-12 or IL-27 and variants thereof, where each of the subunits is fused to either a variable heavy (“VH”) or a variable light (“VL”) regions of scFv or antigen binding fragment obtained from a monoclonal antibody, and a second cytokine, wherein the second cytokine is linked in between the VH and VL regions of the scFv or antigen binding fragment. In certain embodiments, the first cytokine is any multi-subunit cytokine, such as but not limited to IL-10, IL-12 or IL-27, or functional variants thereof that include one or more amino acid substitution(s) that enhance the function of IL-10, IL-12 or IL-27. The fusion protein further includes a second cytokine, which is a cytokine that works in tandem with the multi-subunit cytokine (e.g., IL-10, IL-12 or IL-27) such that there is an additive or synergistic effect when the first and second cytokines are targeted to a specific antigen by the VH and VL regions of the scFv or antigen binding fragment. These second cytokines may be any cytokine, which includes, amongst others, IL-6, IL-4, IL-1, IL-2, IL-3, IL-5, IL-7, IL-8, IL-9, IL-10, IL-15, IL-21 IL-26, IL-27, IL-28, IL-29, GM-CSF, G-CSF, interferons-α, -β, -γ, TGF-β, or tumor necrosis factors-α, -β, basic FGF, EGF, PDGF, IL-4, IL-11, or IL-13. The dual cytokine fusion protein may also include engraftment or replacement of the complement determining regions (“CDRs”) of the scFv with CDRs from any targeting antibody that directs the dual cytokine fusion protein to a target antigen.

In yet another aspect, the present disclosure relates to a dual cytokine fusion protein of formula (Ia) or (Ib):

NH₂—(R¹)—(X¹)—(Zₙ)—(X²)—(R²)—COOH (Formula Ia);

NH₂—(R²)—(X¹)—(Zₙ)—(X²)—(R¹)—COOH (Formula Ib);

wherein
“R¹” is an alpha subunit from any multi-subunit first cytokine, preferably either IL-12-alpha subunit (p35) or IL-27 alpha subunit (p28), more preferably a subunit of SEQ ID No: 1 or 5 or 17 or 19;
“R²” is a beta subunit from any multi-subunit first cytokine, preferably either IL-12-beta subunit (p40) or IL-27 beta subunit (EBI3), more preferably a subunit of SEQ ID No: 3 or 7 or 18 or 20;
wherein when R¹ is an alpha subunit of the first cytokine, R² is a beta subunit of the first cytokine; or when R¹ is p35, R² is p40; or when R¹ is p28, R² is EBI3; or when R¹ is SEQ ID No: 1, 17, or 19, R² is SEQ ID No: 3, 18, or 20; or when R¹ is SEQ ID No: 5, R² is SEQ ID No: 7;
“X¹” is a VL or VH region obtained from a first monoclonal antibody; “X²” is a VH or VL region obtained from the first monoclonal antibody; wherein when X¹ is a VL, X² is a VH or when X¹ is a VH, X² is a VL;
“Z” is any cytokine that enhances the biological function of the multi-subunit cytokine, preferably IFNα-2a, IL-28, IL-29; and
“n” is an integer selected from 0-2.
   In one embodiment, the VH and VL is in the form of a scFv obtained from a human anti-ebola antibody. In another embodiment, the 6 CDRs (CDRs 1-3 from the VH and CDRs 1-3 from the VL) of the scFv obtained from the human anti-ebola antibody are replaced or engrafted with 6 CDR from a second monoclonal antibody that allows the dual cytokine fusion protein to be directed to a specific target, such as, but not limited to enzymes, receptors, extracellular proteins, or intracellular protein, such as those associated with a tumors (e.g., tumor associated antigens (TAAs)), inflammatory response, or autoimmune diseases. The second antibody may include, but not limited to, epidermal growth factor receptor (EGFR); CD14; CD52; various immune check point targets, such as but not limited to PD-L1, PD-1, TIM3, BTLA, LAG3 or CTLA4; CD19; CD20; CD22; CD47; CD123; GD-2; VEGFR1; VEGFR2; HER2; PDGFR; EpCAM; ICAM (ICAM-1, -2, -3, -4, -5), VCAM, FAPα; 5T4; Trop2; EDB-FN; TGFβ Trap; MAdCAM, β7 integrin subunit; α4β7 integrin; α4 integrin; BCMA; PSA; PSMA; CEA; GPC3; SR-A1; SR-A3; SR-A4; SR-A5; SR-A6; SR-B; dSR-C1; SR-D1; SR-E1; SR-F1; SR-F2; SR-G; SR-H1; SR-H2; SR-I1; or SR-J1.

In yet another aspect, the present disclosure relates to a dual cytokine fusion protein comprising IL-12, said fusion protein being Formula (IIa) or (IIb):

NH₂-(p35)-(L)-(X¹)-(L)-(Zₙ)-(L)-(X²)-(L)-(ₚ40)-COOH (Formula IIa);

NH₂-(p40)-(L)-(X¹)-(L)-(Zₙ)-(L)-(X²)-(L)-(p35)-COOH (Formula IIb);
wherein
“p35” is an alpha subunit of IL-12 having a sequence of SEQ ID No; 1, 17 or 19;
“p40” is a beta alpha subunit of IL-12 having a sequence of SEQ ID No; 3, 18, or 20;
“L” is any linker;
X¹″ is a VL or VH region obtained from a first monoclonal antibody; “X²” is a VH or VL region obtained from the first monoclonal antibody; wherein when X¹ is a VL, X² is a VH or when X¹ is a VH, X² is a VL;
“Z” is a cytokine selected from IL-6, IL-4, IL-1, IL-2, IL-3, IL-5, IL-7, IL-8, IL-9, IL-10 monomer, IL-15, IL-21 IL-26, IL-27, IL-28, IL-29, GM-CSF, G-CSF, interferons-α, -β, -γ, TGF-β, or tumor necrosis factors-α, -β, basic FGF, EGF, PDGF, IL-4, IL-11, or IL-13; and
“n” is an integer selected from 0-2.
   In one embodiment, the VH and VL is in the form of a scFv obtained from a human anti-ebola antibody. In another embodiment, the 6 CDRs (CDRs 1-3 from the VH and CDRs 1-3 from the VL) of the scFv obtained from the human anti-ebola antibody are replaced or engrafted with 6 CDR from a second monoclonal antibody that allows the dual cytokine fusion protein to be directed to a specific target, such as, but not limited to enzymes, receptors, extracellular proteins, or intracellular protein, such as those associated with a tumors (e.g., tumor associated antigens (TAAs)), inflammatory response, or autoimmune diseases. The second antibody may include, but not limited to, epidermal growth factor receptor (EGFR); CD14; CD52; various immune check point targets, such as but not limited to PD-L1, PD-1, TIM3, BTLA, LAG3 or CTLA4; CD19; CD20; CD22; CD47; CD123; GD-2; VEGFR1; VEGFR2; HER2; PDGFR; EpCAM; ICAM (ICAM-1, -2, -3, -4, -5), VCAM, FAPα; 5T4; Trop2; EDB-FN; TGFβ Trap; MAdCAM, β7 integrin subunit; α4β7 integrin; α4 integrin; BCMA; PSA; PSMA; CEA; GPC3; SR-A1; SR-A3; SR-A4; SR-A5; SR-A6; SR-B; dSR-C1; SR-D1; SR-E1; SR-F1; SR-F2; SR-G; SR-H1; SR-H2; SR-I1; or SR-J1.

In yet another aspect, the present disclosure relates to a dual cytokine fusion protein comprising IL-27, said fusion protein being Formula (IIIa) or (IIIb):

NH₂-(p28)-(L)-(X¹)-(L)-(Zₙ)-(L)-(X²)-(L)-(EBI3)-COOH (Formula IIIa);

NH₂-(EBI3)-(L)-(X¹)-(L)-(Zₙ)-(L)-(X²)-(L)-(p28)-COOH (Formula IIIb);
wherein
“p28” is an alpha subunit of IL-27 having a sequence of SEQ ID No; 5;
“EBI3” is a beta alpha subunit of IL-27 having a sequence of SEQ ID No; 7;
“L” is any linker;
X¹″ is a VL or VH region obtained from a first monoclonal antibody; “X²” is a VH or VL region obtained from the first monoclonal antibody; wherein when X¹ is a VL, X² is a VH or when X¹ is a VH, X² is a VL;
“Z” is a cytokine selected from IL-6, IL-4, IL-1, IL-2, IL-3, IL-5, IL-7, IL-8, IL-9, IL-10 monomer, IL-15, IL-21 IL-26, IL-27, IL-28, IL-29, GM-CSF, G-CSF, interferons-α, -β, -γ, TGF-β, or tumor necrosis factors-α, -β, basic FGF, EGF, PDGF, IL-4, IL-11, or IL-13; and
“n” is an integer selected from 0-2.
   In one embodiment, the VH and VL is in the form of a scFv obtained from a human anti-ebola antibody. In another embodiment, the 6 CDRs (CDRs 1-3 from the VH and CDRs 1-3 from the VL) of the scFv obtained from the human anti-ebola antibody are replaced or engrafted with 6 CDR from a second monoclonal antibody that allows the dual cytokine fusion protein to be directed to a specific target, such as, but not limited to enzymes, receptors, extracellular proteins, or intracellular protein, such as those associated with a tumors (e.g., tumor associated antigens (TAAs)), inflammatory response, or autoimmune diseases. The second antibody may include, but not limited to, epidermal growth factor receptor (EGFR); CD14; CD52; various immune check point targets, such as but not limited to PD-L1, PD-1, TIM3, BTLA, LAG3 or CTLA4; CD19; CD20; CD22; CD47; CD123; GD-2; VEGFR1; VEGFR2; HER2; PDGFR; EpCAM; ICAM (ICAM-1, -2, -3, -4, -5), VCAM, FAPα; 5T4; Trop2; EDB-FN; TGFβ Trap; MAdCAM, β7 integrin subunit; α4β7 integrin; α4 integrin; BCMA; PSA; PSMA; CEA; GPC3; SR-A1; SR-A3; SR-A4; SR-A5; SR-A6; SR-B; dSR-C1; SR-D1; SR-E1; SR-F1; SR-F2; SR-G; SR-H1; SR-H2; SR-I1; or SR-J1.

In yet another aspect, the present disclosure relates to a dual cytokine fusion protein comprising two multi-subunit proteins, said fusion protein being Formula (IV):

NH₂—(R¹)-(Lₐ)-(X¹)-(Lₐ)-(W¹)-(L_{b})-(W²)-(Lₐ)-(X²)-(Lₐ)-(R²)—COOH (Formula IV);
wherein
“R¹” is an alpha subunit of a first cytokine, such as IL-12 or IL-27 or a first monomer of a homodimeric cytokine, such as IL-10, wherein R¹ is preferably p40;
“R²” is a beta alpha subunit of the first cytokine, such as IL-12 or IL-27 or a second monomer of the homodimeric cytokine, such as IL-10, wherein R² is preferably p35;
“Lₐ” is any linker; preferably (GGGGS)₄ of SEQ ID No.: 45, or (GGGGS)₅ of SEQ ID No.: 44;
“L_{b}” is any linker; preferably GGGSGGG of SEQ ID No.: 43 or of (GGGGS)₃ of SEQ ID No.: 46;
X¹″ is a VL or VH region obtained from a first monoclonal antibody; “X²” is a VH or VL region obtained from the first monoclonal antibody; wherein when X¹ is a VL, X² is a VH or when X¹ is a VH, X² is a VL;
“W¹” is an alpha subunit of a first cytokine, such as IL-12 or IL-27 or a first monomer of a homodimeric cytokine, such as IL-10, preferably a first monomer of IL-10;
“W²” is a beta alpha subunit of the first cytokine, such as IL-12 or IL-27 or a second monomer of the homodimeric cytokine, such as IL-10, preferably a second monomer of IL-10.
   In one embodiment, the VH and VL is in the form of a scFv obtained from a human anti-ebola antibody. In another embodiment, the 6 CDRs (CDRs 1-3 from the VH and CDRs 1-3 from the VL) of the scFv obtained from the human anti-ebola antibody are replaced or engrafted with 6 CDR from a second monoclonal antibody that allows the dual cytokine fusion protein to be directed to a specific target, such as, but not limited to enzymes, receptors, extracellular proteins, or intracellular protein, such as those associated with a tumors (e.g., tumor associated antigens (TAAs)), inflammatory response, or autoimmune diseases. The second antibody may include, but not limited to, epidermal growth factor receptor (EGFR); CD14; CD52; various immune check point targets, such as but not limited to PD-L1, PD-1, TIM3, BTLA, LAG3 or CTLA4; CD19; CD20; CD22; CD47; CD123; GD-2; VEGFR1; VEGFR2; HER2; PDGFR; EpCAM; ICAM (ICAM-1, -2, -3, -4, -5), VCAM, FAPα; 5T4; Trop2; EDB-FN; TGFβ Trap; MAdCAM, β7 integrin subunit; α4β7 integrin; α4 integrin; BCMA; PSA; PSMA; CEA; GPC3; SR-A1; SR-A3; SR-A4; SR-A5; SR-A6; SR-B; dSR-C1; SR-D1; SR-E1; SR-F1; SR-F2; SR-G; SR-H1; SR-H2; SR-I1; or SR-J1.

In yet another aspect, the present disclosure relates to a dual cytokine fusion protein comprising two multi-subunit proteins, said fusion protein being Formula (V):

NH₂-(P35)-(Lₐ)-(X¹)-(Lₐ)-(IL10ₘₒₙₒₘₑᵣ)-(L_{b})-(IL10ₘₒₙₒₘₑᵣ)-(Lₐ)-(X²)-(Lₐ)-(P40)-COOH (Formula Va);

NH₂-(P40)-(Lₐ)-(X¹)-(Lₐ)-(IL10ₘₒₙₒₘₑᵣ)-(L_{b})-(IL10ₘₒₙₒₘₑᵣ)-(Lₐ)-(X²)-(Lₐ)-(P35)-COOH (Formula Vb);
wherein
“p35” is an alpha subunit of IL-12 having a sequence of SEQ ID No: 1, 17, or 19;
“p40” is a beta alpha subunit of IL-12 having a sequence of SEQ ID No; 3, 18, or 20;
“Lₐ” is any linker; preferably (GGGGS)₄ of SEQ ID No.: 45, or (GGGGS)₅ of SEQ ID No.: 44;
“L_{b}” is any linker; preferably GGGSGGG of SEQ ID No.: 43 or (GGGGS)₃ of SEQ ID No.: 46;
X¹″ is a VL or VH region obtained from a first monoclonal antibody; “X²” is a VH or VL region obtained from the first monoclonal antibody; wherein when X¹ is a VL, X² is a VH or when X¹ is a VH, X² is a VL;
“IL10ₘₒₙₒₘₑᵣ” is monomer of IL-10 having a sequence of SEQ ID No: 1, 3, 5, 7, 14, or 16, preferably SEQ ID No: 16;
   In one embodiment, the VH and VL is in the form of a scFv obtained from a human anti-ebola antibody. In another embodiment, the 6 CDRs (CDRs 1-3 from the VH and CDRs 1-3 from the VL) of the scFv obtained from the human anti-ebola antibody are replaced or engrafted with 6 CDR from a second monoclonal antibody that allows the dual cytokine fusion protein to be directed to a specific target, such as, but not limited to enzymes, receptors, extracellular proteins, or intracellular protein, such as those associated with a tumors (e.g., tumor associated antigens (TAAs)), inflammatory response, or autoimmune diseases. The second antibody may include, but not limited to EGFR; CD14; CD52; various immune check point targets, such as but not limited to PD-L1, PD-1, TIM3, BTLA, LAG3 or CTLA4; CD19; CD20; CD22; CD47; CD123; GD-2; VEGFR1; VEGFR2; HER2; PDGFR; EpCAM; ICAM (ICAM-1, -2, -3, -4, -5), VCAM, FAPα; 5T4; Trop2; EDB-FN; TGFβ Trap; MAdCAM, β7 integrin subunit; α4β7 integrin; α4 integrin; BCMA; PSA; PSMA; CEA; GPC3; SR-A1; SR-A3; SR-A4; SR-A5; SR-A6; SR-B; dSR-C1; SR-D1; SR-E1; SR-F1; SR-F2; SR-G; SR-H1; SR-H2; SR-I1; or SR-J1.

In other aspects, the present disclosure relates to a nucleic acid molecule that encodes the multi-subunit dual cytokine fusion protein. These would include those that encode the dual cytokine fusion protein represented by formula (Ia), (Ib), (IIa), (IIb), (IIIa), (IIIb), (IV), (Va) and (Vb)

In other aspects, the present disclosure relates to methods of making and purifying the dual cytokine fusion protein. In one embodiment, the method of making the dual cytokine fusion protein includes recombinantly expressing the nucleic acid encoding the dual cytokine fusion protein.

In other aspects, the present disclosure relates to a method of treating cancer comprising administering to a subject in need thereof, an effective amount of the dual cytokine fusion protein.

In other aspects, the present disclosure relates to a method of treating inflammatory diseases or conditions comprising administering to a subject in need thereof, an effective amount of the dual cytokine fusion protein. Preferably, the inflammatory disease is Crohn's disease, psoriasis, and/or rheumatoid arthritis.

In other aspects, the present disclosure relates to a method of treating immune diseases or conditions comprising administering to a subject in need thereof, an effective amount of the dual cytokine fusion protein.

In other aspects, the present disclosure relates to method of treating, inhibiting, and/or alleviating sepsis and/or septic shock and associated symptoms thereof.

The above simplified summary of representative aspects serves to provide a basic understanding of the present disclosure. This summary is not an extensive overview of all contemplated aspects, and is intended to neither identify key or critical elements of all aspects nor delineate the scope of any or all aspects of the present disclosure. Its sole purpose is to present one or more aspects in a simplified form as a prelude to the more detailed description of the disclosure that follows. To the accomplishment of the foregoing, the one or more aspects of the present disclosure include the features described and exemplarily pointed out in the claims.

### BRIEF DESCRIPTION OF THE DRAWINGS

FIG. 1 is a schematic diagram of a dual cytokine fusion protein in folded and linear form.

FIG. 2 is a schematic diagram that is representative of a dual cytokine fusion protein embodied in the present disclosure, wherein the dual cytokine fusion protein comprises terminally linked IL-12α (p35) and IL-12β (p40) subunits, where a second cytokine is incorporated into the linker of a scFv between the VH and VL from an anti-X antibody (e.g., from a human anti-ebola antibody). The 6 CDRs (3 from the VH and 3 from the VL) may be optionally substituted or engrafted with 6 CDRs from a second antibody (e.g., such as those that target TAAs, such as an anti-HER2, anti-EGFR, anti-VEGFR1, or anti-VEGFR2 antibody).

FIG. 3 is a schematic diagram that is representative of a dual cytokine fusion protein embodied in the present disclosure, wherein the dual cytokine fusion protein comprises terminally linked IL-27α (p28) and IL-27β (EBI3) subunits, where a second cytokine is incorporated into the linker of a scFv between the VH and VL from an anti-X antibody (e.g., from a human anti-ebola antibody). The 6 CDRs (3 from the VH and 3 from the VL) may be optionally substituted or engrafted with 6 CDRs from a second antibody (e.g., such as those that target TAAs, such as, but not limited to, an anti-HER2, an anti-HER3, anti-EGFR, anti-VEGFR1, or anti-VEGFR2 antibody).

FIG. 4 is a schematic diagram that is representative of a dual cytokine fusion protein comprising IL-27 and IL-28 termed “DK27²⁸”.

FIG. 5 is a schematic diagram that is representative of a dual cytokine fusion protein comprising IL-27 and IL-29 termed “DK27²⁹”.

FIG. 6 is a schematic diagram representing one of the previously disclosed IL-10 fusion protein constructs where IL-10 monomers are terminally linked to a scaffolding comprising a scFv described in U.S. Pat. No. 10,858,412.

FIG. 7 is a schematic diagram that is a representative examples of a dual cytokine fusion protein comprising two multi-subunit (or dimeric) cytokines, in particular, a dual cytokine fusion protein comprising IL-12 and IL-10 termed DK12¹⁰.

FIG. 8 is a schematic diagram that is a representative examples of a dual cytokine fusion protein comprising two multi-subunit (or dimeric) cytokines, in particular, a dual cytokine fusion protein comprising IL-27 and IL-10 termed DK27¹⁰.

FIG. 9 is a graph showing that DK12¹⁰ (EGFR) using standard linkers, such as (GGGGS)₃, between IL-10 and the scFv and between IL-12 and the scFv have partial cytolytic effect on target cancer cells in combination with BiTE.

FIG. 10 is a graph showing that DK12¹⁰ (EGFR) using extended linkers between IL-10 and the scFv and between IL12 and the scFv have improved cytolytic effect on target cancer cells in combination with BiTE.

### DETAILED DESCRIPTION

Exemplary aspects are described herein in the context of a dual cytokine fusion protein comprising a multi-subunit first cytokine (such as IL-12 or IL-27), methods of making the dual cytokine fusion protein comprising a multi-subunit first cytokine (such as IL-12 or IL-27), and methods of using the dual cytokine fusion protein comprising a multi-subunit first cytokine (such as IL-12 or IL-27) for treating inflammatory diseases or conditions, immune diseases or conditions, treating and/or preventing cancer. Those of ordinary skill in the art will realize that the following description is illustrative only and is not intended to be in any way limiting. Other aspects will readily suggest themselves to those skilled in the art having the benefit of this disclosure. Reference will now be made in detail to implementations of the exemplary aspects as illustrated in the accompanying drawings. The same reference indicators will be used to the extent possible throughout the drawings and the following description to refer to the same or like items.

Although a number of methods and materials similar or equivalent to those described herein can be used in the practice of the various described embodiments, the preferred materials and methods are described herein.

Unless otherwise indicated, the embodiments described herein employ conventional methods and techniques of molecular biology, biochemistry, pharmacology, chemistry, and immunology, well known to a person skilled in the art. Many of the general techniques for designing and fabricating the dual cytokine fusion proteins comprising the multi-subunit first cytokine (such as IL-12 or IL-27), as well as the assays for testing the expression and function of dual cytokine fusion proteins comprising the multi-subunit first cytokine (such as IL-12 or IL-27), are well known methods that are readily available and detailed in the art. See, e.g., Sambrook, et al., Molecular Cloning: A Laboratory Manual (2nd Edition, 1989); Methods In Enzymology (S. Colowick and N. Kaplan eds., Academic Press, Inc.); Handbook of Experimental Immunology, Vols. I-IV (D. M. Weir and C. C. Blackwell eds., Blackwell Scientific Publications); A. L. Lehninger, Biochemistry (Worth Publishers, Inc., current addition). N-terminal aldehyde based PEGylation chemistry is also well known in the art.

### Definitions

The following terms will be used to describe the various embodiments discussed herein, and are intended to be defined as indicated below.

As used herein in describing the various embodiments, the singular forms “a”, “an” and “the” include plural referents unless the content clearly dictates otherwise.

The term “about”, refers to a deviance of between 0.0001-5% from the indicated number or range of numbers. In one embodiment, the term “about”, refers to a deviance of between 1-10% from the indicated number or range of numbers. In one embodiment, the term “about”, refers to a deviance of up to 25% from the indicated number or range of numbers. In a more specific embodiment, the term “about” refers to a difference of 1-25% in terms of nucleotide sequence homology or amino acid sequence homology when compared to a wild-type sequence.

The term “multi-subunit cytokine” refers to a cytokine protein comprising at least an alpha subunit and a beta subunit to make a heterodimer or two monomers to make a homodimer. As reference, a multi-subunit cytokine may include, amongst other, IL-10, IL-12 or IL-27. Other multi-subunit cytokines are known by those of skill in the art and may be substituted into the terminal ends of the dual cytokine fusion proteins described herein.

The term “interleukin-12” or “IL-12” refers to a protein comprising an alpha (p35) and beta (p40) subunit, non-covalently joined to form a heterodimer. As used herein, unless otherwise indicated “interleukin-12” and “IL-12” refers to any form of IL-12, including but not limited to human; mouse, or variant forms. For example, the term “wild-type” or “native” would thus correspond to an amino acid sequence that is most commonly found in nature for the alpha and beta subunits. In one embodiment, p35 is a sequence of SEQ ID No: 1, 17 or 19 and p40 is a sequence of SEQ ID No: 3, 18, or 20.

The term “interleukin-27” or “IL-27” refers to a protein comprising an alpha (p28) and beta (EBI3) subunit, non-covalently joined to form a heterodimer. As used herein, unless otherwise indicated “interleukin-12” and “IL-12” refers to any form of IL-12, including but not limited to human; mouse, or variant forms. For example, the term “wild-type” or “native” would thus correspond to an amino acid sequence that is most commonly found in nature for the alpha and beta subunits. In one embodiment, p28 is a sequence of SEQ ID No: 5 and EBI3 is a sequence of SEQ ID No: 7.

The term “interleukin-10” or “IL-10” refers to a protein comprising two monomers that joined to form a homodimer. As used herein, unless otherwise indicated “interleukin-10” and “IL-10” refers to any form of IL-10, including but not limited to human; mouse, or variant forms. For example, the term “wild-type” or “native” would thus correspond to an amino acid sequence that is most commonly found in nature for the alpha and beta subunits. In one embodiment, human IL-10 is a sequence of SEQ ID No: 31, mouse IL-10 is a sequence of SEQ ID No: 37, viral forms of IL-10 include EBV IL-10 having SEQ ID No: 33, and CMV IL-10 having SEQ ID No: 35.

The terms “variant,” “analog” and “mutein” refer to biologically active derivatives of the reference molecule, that retain a desired activity, such as, for example, anti-inflammatory activity. Generally, the terms “variant,” “variants,” “analog” and “mutein” as it relates to a polypeptide refers to a compound or compounds having a native polypeptide sequence and structure with one or more amino acid additions, substitutions (which may be conservative in nature), and/or deletions, relative to the native molecule. As such, the terms “IL-12 variant”, “variant IL-12,” “IL-12 variant molecule,” “IL-27 variant”, “variant IL-27,” “IL-27 variant molecule,” and grammatical variations and plural forms thereof are all intended to be equivalent terms that refer to an IL-12 or IL-27 amino acid (or nucleic acid) sequence that differs from wild-type IL-12 or IL-27. The difference in amino acid sequence for IL-12 or IL-27 may be additions, deletions, or substitutions within the alpha, beta, or both subunits such that there is anywhere from 1-25% in sequence identity or homology. These variant forms include modifications to the glycosylation (deglycosylated or aglycosylated) forms thereof to the protein. IL-10 variant forms may include those have increased or decreased binding affinity when compared to wild-type IL-10. Thus in one embodiment, a variant form of IL-10 having increased or higher binding affinity includes an IL-10 variant (internally denoted as DV07) of SEQ ID No.:41 or an IL-10 variant having decreased or lower binding affinity (internally denoted as DV06) of SEQ ID No: 39.

The term “fusion protein” refers to a combination or conjugation of two or more proteins or polypeptides that results in a novel arrangement of proteins that do not normally exist naturally. The fusion protein is a result of covalent linkages of the two or more proteins or polypeptides. The two or more proteins that make up the fusion protein may be arranged in any configuration from amino-terminal end (“NH₂”) to carboxy-terminal end (“COOH”). Thus, for example, the carboxy-terminal end of one protein may be covalently linked to either the carboxy terminal end or the amino terminal end of another protein. Exemplary fusion proteins may include combining (from N-terminal to C-terminal) an alpha subunit of IL-12 to an antibody VH domain to a second cytokine (such as IFN-alpha, IL-28, or IL-29) to a VL domain (such that the VH and VL domains form a VH/VL pair) to a beta subunit of IL-12. Another exemplary fusion protein may include combining (from N-terminal to C-terminal) an alpha subunit of IL-27 to an antibody VH domain to a second cytokine (such as IFN-alpha, IL-28, or IL-29) to a VL domain (such that the VH and VL domains form a VH/VL pair) to a beta subunit of IL-27. Yet another exemplary fusion protein may include combining (from N-terminal to C-terminal) an alpha subunit of IL-12 to an antibody VH domain to a first monomer of a homodimeric cytokine (such as IL-10) to a second monomer of the homodimeric cytokine (such as IL-10) to a VL domain (such that the VH and VL domains form a VH/VL pair) to a beta subunit of IL-12. In one preferred embodiment, a representative forms of a multi-subunit dual cytokine fusion protein may include heterodimeric cytokines such as IL-12 or IL-27 in combination with monomeric cytokines such as IL-6, IL-4, IL-1, IL-2, IL-3, IL-5, IL-7, IL-8, IL-9, IL-15, IL-21 IL-26, IL-27, IL-28, IL-29, GM-CSF, G-CSF, interferons-α, -β, -γ, TGF-β, or tumor necrosis factors-α, -β, basic FGF, EGF, PDGF, IL-4, IL-11, or IL-13. In another embodiment, a multi-subunit dual cytokine fusion protein may include a heterodimer cytokine such as IL-12 or IL-27 in combination with a homodimeric cytokine such as IL-10, IL-10 variants, mouse IL-10, DV07 (SEQ ID No:41), or DV06 (SEQ ID No:39).

The term “homolog,” “homology,” “homologous” or “substantially homologous” refers to the percent identity between at least two polynucleotide sequences or at least two polypeptide sequences. Sequences are homologous to each other when the sequences exhibit at least about 50%, preferably at least about 75%, more preferably at least about 80%-85%, preferably at least about 90%, and most preferably at least about 95%-98% sequence identity over a defined length of the molecules.

The term “sequence identity” refers to an exact nucleotide-by-nucleotide or amino acid-by-amino acid correspondence. The sequence identity may range from 100% sequence identity to 50% sequence identity. A percent sequence identity can be determined using a variety of methods including but not limited to a direct comparison of the sequence information between two molecules (the reference sequence and a sequence with unknown percent identity to the reference sequence) by aligning the sequences, counting the exact number of matches between the two aligned sequences, dividing by the length of the reference sequence, and multiplying the result by 100. Readily available computer programs can be used to aid in the identification of percent identity.

The terms “subject,” “individual” or “patient” are used interchangeably herein and refer to a vertebrate, preferably a mammal. Mammals include, but are not limited to, murine, rodent, simian, human, farm animals, sport animals, and certain pets.

The term “administering” includes routes of administration which allow the active ingredient of the application to perform their intended function.

A “therapeutically effective amount” as it relates to, for example, administering the dual cytokine fusion proteins described herein, refers to a sufficient amount of dual cytokine fusion proteins to promote certain biological activities. These might include, for example, suppression of myeloid cell function, enhanced Kupffer cell activity, and/or lack of any effect on CD8⁺ T cells or enhanced CD8⁺ T-cell activity as well as blockade of mast cell upregulation of Fc receptor or prevention of degranulation. Thus, an “effective amount” will ameliorate or prevent a symptom or sign of the medical condition. Effective amount also means an amount sufficient to allow or facilitate diagnosis.

The term “treat” or “treatment” refers to a method of reducing the effects of a disease or condition. Treatment can also refer to a method of reducing the underlying cause of the disease or condition itself rather than just the symptoms. The treatment can be any reduction from native levels and can be, but is not limited to, the complete ablation of the disease, condition, or the symptoms of the disease or condition.

Dual Cytokine Fusion Protein Structure

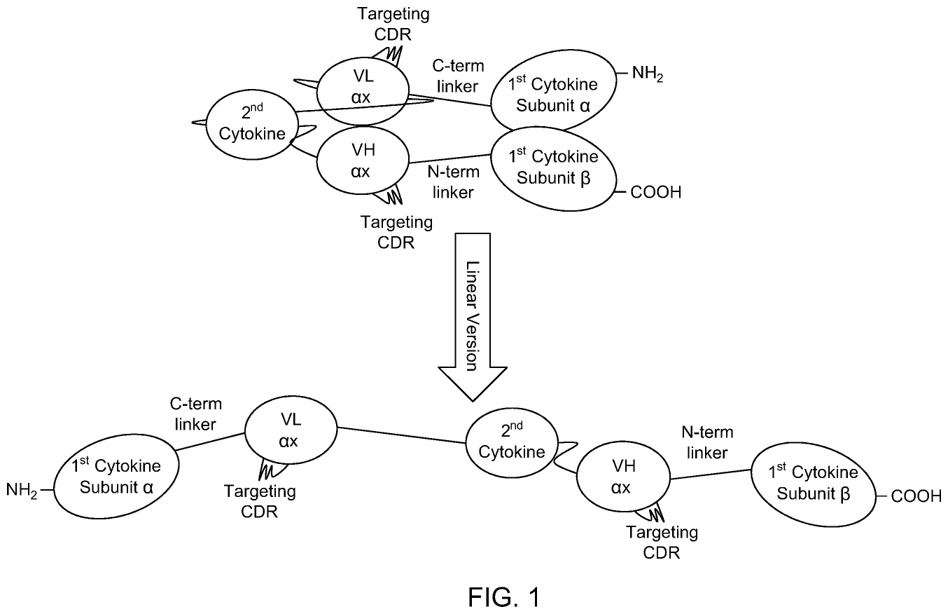

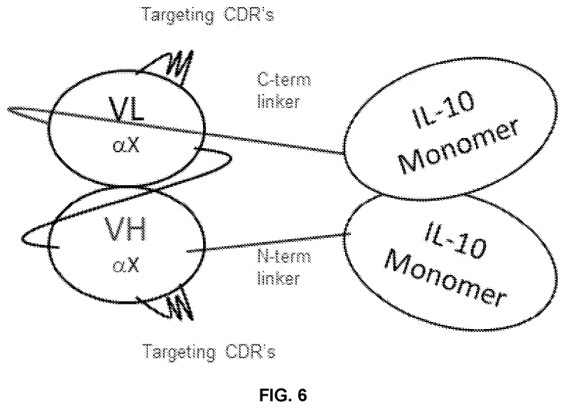

The present disclosure provides an improvement on an embodiment of an IL-10 fusion protein previously described in U.S. Pat. No. 10,858,412 (filed as U.S. application Ser. No. 16/811,718), which is incorporated by reference in its entirety. FIG. 6 is a schematic diagram representing one of the previously disclosed IL-10 fusion protein constructs described in U.S. Pat. No. 10,858,412. FIG. 1 provides a general schematic representation of how the present application improves on the original IL-10 fusion protein. The improvement on the IL-10 fusion protein includes (1) substituting the IL-10 monomers with alpha and beta subunits of a multi-subunit cytokine and (2) incorporating a second cytokine molecule between the VH and VL domains of a scFv (i.e. in the hinge region of the scFv). The dual cytokine fusion protein of the present application may be constructed on a scaffolding system comprising a VH and VL (scFv) featuring an alpha and beta subunit of a multi-subunit cytokine, where the alpha subunit is fused on the N-terminus and the beta subunit is sued to the C-terminus (or vice versa). According the term fused to the “terminal ends” will mean that either an alpha (or beta) subunit will be located on the N-terminal end and the beta (or alpha) subunit will be located on the C-terminal end of the fusion protein. The scaffolding system will comprises a scFv, preferably obtained from an antibody that is specific for HIV or ebola, preferably the scFv is obtained from a human anti-ebola antibody. The dual cytokine fusion protein includes a scFv (preferably obtained from a human anti-ebola antibody) having 6 complementarity-determining regions (“CDRs”), where there are 3 CDRs (CDR 1-3) in the VH and 3 CDRs (CDR 1-3) in the VL. Optionally, the VH and VL regions are capable of targeting the dual cytokine fusion protein to a specific antigen. This may be accomplished by substituting the 6 CDR regions of the VH and VL pair (3 CDRs in the VH and 3 CDRs in the VL) with 6 CDR regions from a VH and VL of a receptor or antigen targeting antibody, or antigen binding fragment thereof. This process is also generally known as CDR grafting or CDR engraftment. Those of skill in the art are capable of substituting and optimizing the engraftment or grafting of the 6 CDR into the scFv framework regions or into scFv scaffolding described herein. These are well known and practiced techniques used by those of skill in the art. The 6 CDR regions from, for example the scFv obtained from a human anti-ebola antibody are substitutable with 6 CDRs from any monoclonal antibody, which any person of skill would be capable of determining based on the specific target of interest. For example, the specific target may include, but not limited to, enzymes, receptors, extracellular proteins, or intracellular protein, such as those associated with a tumors (e.g., tumor associated antigens (TAAs)), inflammatory response, or autoimmune diseases. Thus, the 6 CDRs targeting the specific target may be any antibody, including but not limited to, those that are specific for EGFR; CD14; CD52; various immune check point targets, such as but not limited to PD-L1, PD-1, TIM3, BTLA, LAG3 or CTLA4; CD19; CD20; CD22; CD47; CD123; GD-2; VEGFR1; VEGFR2; HER2; PDGFR; EpCAM; ICAM (ICAM-1, -2, -3, -4, -5), VCAM, FAPα; 5T4; Trop2; EDB-FN; TGFβ Trap; MAdCAM, β7 integrin subunit; α4β7 integrin; α4 integrin; BCMA; PSA; PSMA; CEA; GPC3; SR-A1; SR-A3; SR-A4; SR-A5; SR-A6; SR-B; dSR-C1; SR-D1; SR-E1; SR-F1; SR-F2; SR-G; SR-H1; SR-H2; SR-I1; or SR-J1. The aforementioned list is representative of the possible targeting antibodies that may be incorporated into the dual cytokine fusion protein of the present disclosure, and a person of skill in the art would be able to recognize that the CDRs from other targeting antibodies, especially ones that target surface markers on cancers or inflammatory tissues, may be engrafted into the scaffolding. In a preferred embodiment, the antibody is an anti-HER2, anti-HER3, anti-EGFR, anti-VEGFR1, anti-VEGFR2, anti-BCMA, anti-PSA, anti-PSMA, anti-CD19, anti-CD20, anti-CD22, anti-CEA, anti-GPC3, or anti-CD14 antibody.

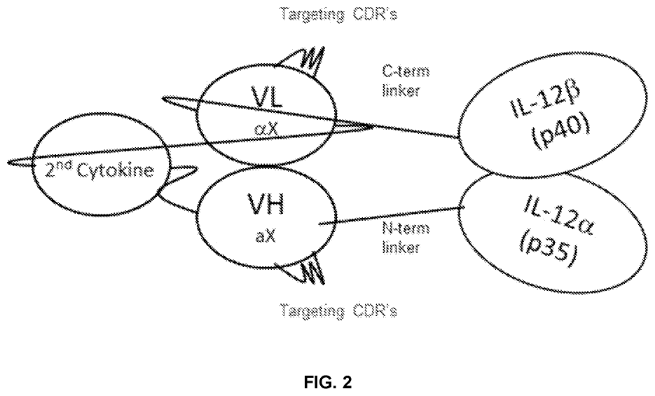

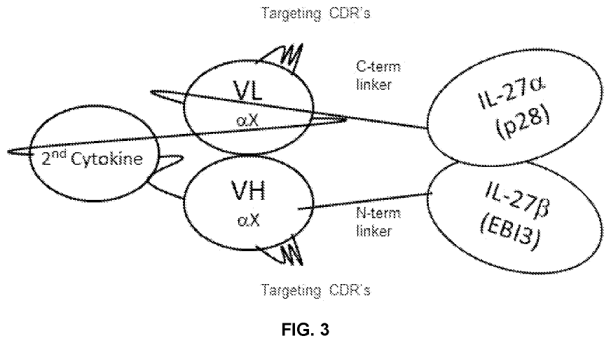

In a first aspect, the present application relates to a dual cytokine fusion protein comprising IL-12 or IL-27 and at least one other cytokine, whereby the dual cytokine fusion protein has a combined or synergistic functionality when compared to IL-12 or IL-27 and the other cytokine fusion individually. FIG. 1 is a representative diagram of the improved dual cytokine fusion protein comprising the alpha and beta subunits of the multi-subunit cytokine fused to the terminal ends of the fusion protein. In particular, the improved dual cytokine fusion protein adapts the same or substantially same scaffolding system made up of a VH and VL scFv whereby alpha and beta subunits of IL-12 or IL-27, for example, terminate the dual fusion protein at the amino and carboxy terminal ends (see, e.g., FIG. 2-5). In certain embodiments the IL-12 subunit is an alpha subunit or p35 of SEQ ID No: 1, 17, or 19 or a beta subunit or p40 of SEQ ID No: 3, 18, or 20 wherein the amino acid subunit fused to the scFv lacks the signal peptide or leader sequence. In other embodiments the IL-27 subunit is an alpha subunit or p28 of SEQ ID No: 5 or a beta subunit of and EBI3 of SEQ ID No: 7, wherein the amino acid fused to the scFv lacks the signal peptide or leader sequence. In certain embodiments, modifications to one or both subunits of IL-12 or IL-27 maybe include additions, deletions, or substitutions. These modifications may include modifications that alter binding affinity (increase or decrease), alter glycosylation sites, or decrease immunogenicity. In certain embodiments, the glycosylation sites in IL-27, preferably in EIB3 are modified, more preferably amino acid positions 55-57 and/or 105-107 of SEQ ID No: 7, even more preferably position 57 and/or 107 of SEQ ID No: 7, where threonine is substituted. The second cytokine is conjugated to the dual cytokine fusion protein by being fused between the VH and VL regions of the scFv, which is the hinge region of the scFv (see, e.g. FIG. 1-5). The dual cytokine fusion protein is capable of forming a functional protein complex whereby the alpha and beta subunits heterodimerize along with the pairing of the VH and VL regions to form a pair that associate together to form a scFv complex.

In certain embodiments, the dual cytokine fusion protein comprising the multi-subunit cytokine is a structure having formula Ia or Ib

NH₂—(R¹)—(X¹)—(Zₙ)—(X²)—(R²)—COOH (Formula Ia);

NH₂—(R²)—(X¹)—(Zₙ)—(X²)—(R¹)—COOH (Formula Ib)

wherein
“R¹” is an alpha subunit of a first cytokine sequence selected from SEQ ID No: 1, 17, 19 or 5;
“R²” is a beta subunit of a first cytokine sequence selected from SEQ ID No: 3, 18, 20, or 7;
   wherein when R¹ is SEQ ID No: 1, 17 or 19, R² is SEQ ID No: 3, 18, or 20 or when R¹ is SEQ ID No: 5, R² is SEQ ID No:7;
“X¹” is a VL or VH region obtained from a first monoclonal antibody;
“X²” is a VH or VL region obtained from the first monoclonal antibody;
   wherein when X¹ is a VL, X² is a VH or when X¹ is a VH, X² is a VL
“Z” is a cytokine;
“n” is an integer selected from 0-2.

In another embodiment, the dual cytokine fusion protein is a structure having formula IIa or IIb

NH₂-(p35)-(L)-(X¹)-(L)-(Zₙ)-(L)-(X²)-(L)-(p40)-COOH (Formula IIa);

NH₂-(p40)-(L)-(X¹)-(L)-(Zₙ)-(L)-(X²)-(L)-(p35)-COOH (Formula IIb);

wherein
“p35” is a sequence of SEQ ID No: 1, 17, or 19;
“p40” is a sequence of SEQ ID No: 3, 18, or 20;
“L” is a linker;
“X¹” is a VL or VH region obtained from a first monoclonal antibody;
“X²” is a VH or VL region obtained from the first monoclonal antibody;
   wherein when X¹ is a VL, X² is a VH or when X¹ is a VH, X² is a VL;
“Z” is a cytokine selected from IL-6, IL-4, IL-1, IL-2, IL-3, IL-5, IL-7, IL-8, IL-9, IL-15, IL-21, IL-26, IL-27, IL-28, IL-29, GM-CSF, G-CSF, interferons-α, -β, -γ, TGF-β, or tumor necrosis factors-α, -β, basic FGF, EGF, PDGF, IL-4, IL-11, or IL-13;
“n” is an integer selected from 0-2.

In another embodiment, the dual cytokine fusion protein is a structure having formula IIIa or IIIb

NH₂-(p28)-(L)-(X¹)-(L)-(Zₙ)-(L)-(X²)-(L)-(EBI3)-COOH (Formula IIIa);

NH₂-(EBI3)-(L)-(X¹)-(L)-(Zₙ)-(L)-(X²)-(L)-(p28)-COOH (Formula IIIb);

wherein
“p28” is a sequence of SEQ ID No: 5;
“EBI3” is a sequence of SEQ ID No: 7;
“L” is a linker;
“X¹” is a VL or VH region obtained from a first monoclonal antibody;
“X²” is a VH or VL region obtained from the first monoclonal antibody;
   wherein when X¹ is a VL, X² is a VH or when X¹ is a VH, X² is a VL;
“Z” is a cytokine selected from IL-6, IL-4, IL-1, IL-2, IL-3, IL-5, IL-7, IL-8, IL-9, IL-15, IL-21, IL-26, IL-28, IL-29, GM-CSF, G-CSF, interferons-α, -β, -γ, TGF-β, or tumor necrosis factors-α, -β, basic FGF, EGF, PDGF, IL-4, IL-11, or IL-13;
“n” is an integer selected from 0-2.

In another embodiment, the VH and VL regions are from an antibody, antibody fragment, or antigen binding fragment thereof. The antigen binding fragment includes, but is not limited to, a scFv, Fab, F(ab′)₂, V-NAR, diabody, or nanobody. Preferably the VH and VL, are from a single chain variable fragment (“scFv”).

In another embodiment, the dual cytokine fusion protein comprising a first multi-subunit cytokine (e.g., IL-12 or IL-27) includes a VH and VL pair from a single antibody. The VH and VL pair act as a scaffolding onto which first multi-subunit cytokine may be attached such that the alpha and beta subunits of the multi-subunit cytokine may be able to heterodimerize into a functioning cytokine. A person of skill in the art will therefore appreciate that the VH and VL scaffolding used in the fusion protein may be selected based on the desired physical attributes needed for proper heterodimerization of the first multi-subunit cytokine (e.g., IL-12 or IL-27) and/or the desire to maintain VH and VL targeting ability. Likewise, a person of skill will also understand that the 6 CDRs within the VH and VL pair (3 CDRs from the VH and 3 CDRs from VL) may also be substituted with 6 CDRs from other antibodies to obtain a specifically targeted fusion protein. In one embodiment, 3 CDRs from a VH and 3 CDRs from a VL (i.e., a VH and VL pair) of any monoclonal antibody may be engrafted into a scaffolding system. It is also envisioned that if the fusion protein is not intended to target any specific antigen, a VH and VL pair may be selected as the scaffolding that does not target any particular antigen (or is an antigen in low abundance in vivo), such as the VH and VL pair from a human anti-HIV and/or human anti-Ebola antibody. The fusion protein may comprise a range of 1-4 variable regions. In another embodiment, the variable regions may be from the same antibody or from at least two different antibodies. The amino acid sequence encoding the multi-subunit cytokines will be fused to the scFv scaffolding without the signal peptides (or leader sequence).

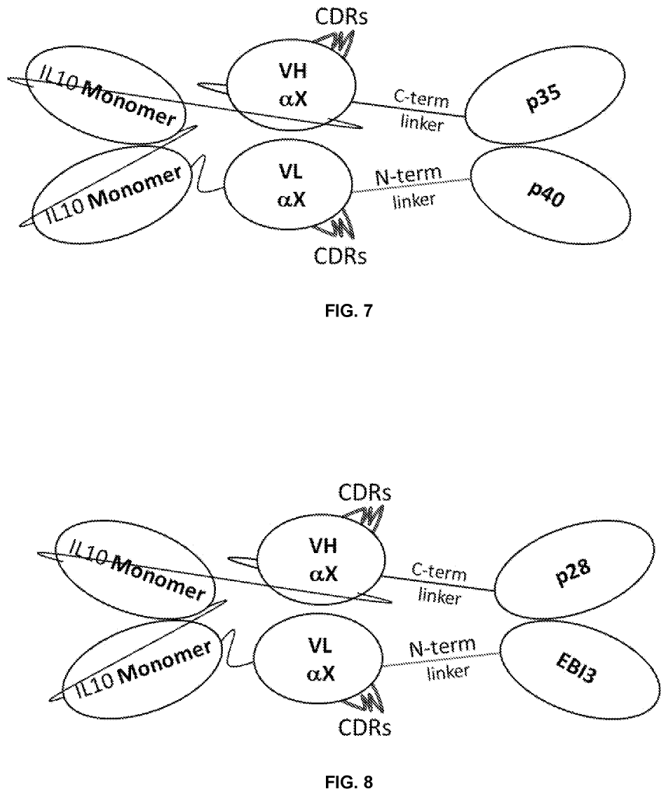

In yet another aspect, the dual cytokine fusion protein may comprise two multi-subunit proteins. For example, the dual cytokine fusion protein may comprise a first cytokine that is a heterodimer (such as but not limited to IL12 or IL27) and then a second homodimeric cytokine (such as but not limited to IL10). The second homodimeric cytokine will be capable of being fused between the VH and VL regions. A representative image of a two multi-subunit dual cytokine fusion protein is provided in FIGS. 7 and 8. Thus, in one embodiment of the invention, said fusion protein will have a generic formula of Formula (IV):

NH₂—(R¹)-(Lₐ)-(X¹)-(L_{b})-(W¹)-(L_{c})-(W²)-(L_{b})-(X²)-(Lₐ)-(R²)—COOH (Formula IV)
wherein
“R¹” is an alpha subunit of a first cytokine, such as IL-12 or IL-27 or a first monomer of a homodimeric cytokine, such as IL-10, preferably (p40);
“R²” is a beta alpha subunit of the first cytokine, such as IL-12 or IL-27 or a second monomer of the homodimeric cytokine, such as IL-10, preferably p35;
“Lₐ” is any linker; preferably (GGGGS)₃ of SEQ ID No: 46, (GGGGS)₄ of SEQ ID No: 45, or (GGGGS)₅ of SEQ ID No: 44;
“L_{b}” is any linker; preferably (GGGGS)₃ of SEQ ID No: 46, (GGGGS)₄ of SEQ ID No: 45, or (GGGGS)₅ of SEQ ID No: 44;
“Lc” is any linker; preferably GGGSGGG of SEQ ID No: 43 or (GGGGS)₃ of SEQ ID No: 46;
X¹″ is a VL or VH region obtained from a first monoclonal antibody; “X²” is a VH or VL region obtained from the first monoclonal antibody; wherein when X¹ is a VL, X² is a VH or when X¹ is a VH, X² is a VL;
“W¹” is an alpha subunit of a first cytokine, such as IL-12 or IL-27 or a first monomer of a homodimeric cytokine, such as IL-10, preferably a first monomer of IL-10 of SEQ ID No: 31, 33, 35, 37, 39, or 41;
“W²” is a beta alpha subunit of the first cytokine, such as IL-12 or IL-27 or a second monomer of the homodimeric cytokine, such as IL-10, preferably a second monomer of IL-10 of SEQ ID No: 31, 33, 35, 37, 39, or 41.
   In one embodiment, the VH and VL is in the form of a scFv obtained from a human anti-ebola antibody. In another embodiment, the 6 CDRs (CDRs 1-3 from the VH and CDRs 1-3 from the VL) of the scFv obtained from the human anti-ebola antibody are replaced or engrafted with 6 CDR from a second monoclonal antibody that allows the dual cytokine fusion protein to be directed to a specific target, such as, but not limited to enzymes, receptors, extracellular proteins, or intracellular protein, such as those associated with a tumors (e.g., tumor associated antigens (TAAs)), inflammatory response, or autoimmune diseases. The second antibody may include, but not limited to EGFR; CD14; CD52; various immune check point targets, such as but not limited to PD-L1, PD-1, TIM3, BTLA, LAG3 or CTLA4; CD19; CD20; CD22; CD47; CD123; GD-2; VEGFR1; VEGFR2; HER2; PDGFR; EpCAM; ICAM (ICAM-1, -2, -3, -4, -5), VCAM, FAPα; 5T4; Trop2; EDB-FN; TGFβ Trap; MAdCAM, β7 integrin subunit; α4β7 integrin; α4 integrin; BCMA; PSA; PSMA; CEA; GPC3; SR-A1; SR-A3; SR-A4; SR-A5; SR-A6; SR-B; dSR-C1; SR-D1; SR-E1; SR-F1; SR-F2; SR-G; SR-H1; SR-H2; SR-I1; or SR-J1. In a more preferred embodiment, the dual cytokine fusion protein comprising two multi-subunit proteins is represented by Formula Va or Vb

NH₂-(P35)-(Lₐ)-(X¹)-(L_{b})-(IL10ₘₒₙₒₘₑᵣ)-(L_{c})-(IL10ₘₒₙₒₘₑᵣ)-(L_{b})-(X²)-(Lₐ)-(P40)-COOH (Formula Va);

NH₂-(P40)-(Lₐ)-(X¹)-(Lₐ)-(IL10ₘₒₙₒₘₑᵣ)-(L_{c})-(IL10ₘₒₙₒₘₑᵣ)-(L_{b})-(X²)-(Lₐ)-(P35)-COOH (Formula Vb);
wherein
“p35” is an alpha subunit of IL-12 having a sequence of SEQ ID No; 1, 17, 19;
“p40” is a beta alpha subunit of IL-12 having a sequence of SEQ ID No; 3, 18, 20;
“Lₐ” is any linker; preferably (GGGGS)₃ of SEQ ID No: 46, (GGGGS)₄ of SEQ ID No: 45, or (GGGGS)₅ of SEQ ID No: 44;
“L_{b}” is any linker; preferably (GGGGS)₃ of SEQ ID No: 46, (GGGGS)₄ of SEQ ID No: 45, or (GGGGS)₅ of SEQ ID No: 44;
“L_{c}” is any linker; preferably GGGSGGG of SEQ ID No: 43 or (GGGGS)₃ of SEQ ID No: 46
X¹″ is a VL or VH region obtained from a first monoclonal antibody; “X²” is a VH or VL region obtained from the first monoclonal antibody; wherein when X¹ is a VL, X² is a VH or when X¹ is a VH, X² is a VL;
“IL10ₘₒₙₒₘₑᵣ” is monomer of IL-10 having a sequence of SEQ ID No: 31, 33, 35, 37, 39, or 41, preferably SEQ ID No: 41.
   the first monoclonal antibody being an anti-ebola antibody (US Published Application 2018/0180614, incorporated by reference in its entirety, especially mAbs described in Tables 2, 3, and 4), which may be engrafted with 6 CDRs from a second antibody having specificity for any one of EGFR; CD14; CD52; various immune check point targets, such as but not limited to PD-L1, PD-1, TIM3, BTLA, LAG3 or CTLA4; CD19; CD20; CD22; CD47; CD123; GD-2; VEGFR1; VEGFR2; HER2; PDGFR; EpCAM; ICAM (ICAM-1, -2, -3, -4, -5), VCAM, FAPα; 5T4; Trop2; EDB-FN; TGFβ Trap; MAdCAM, β7 integrin subunit; α4β7 integrin; α4 integrin; BCMA; PSA; PSMA; CEA; GPC3; SR-A1; SR-A3; SR-A4; SR-A5; SR-A6; SR-B; dSR-C1; SR-D1; SR-E1; SR-F1; SR-F2; SR-G; SR-H1; SR-H2; SR-I1; or SR-J1.

Tables 2a-2d are different combinations of a dual cytokine fusion protein comprising IL-12 and IL-10 as represented by Formula IV.

TABLE 2a
(Embodiments of DK12¹⁰ Combination 1)

| (R¹) | (X¹) | (W¹) | (W²) | (X²) | (R²) |
|---|---|---|---|---|---|
| P40 | VH EGFR | IL10 | IL10 | VL EGFR | P35 |
| | VH HER2 | monomer¹ | monomer¹ | VL HER2 | |
| | VH HER3 | | | VL HER3 | |
| | VH VEGFR2 | | | VL VEGFR2 | |
| | VH VEGFR1 | | | VL VEGFR1 | |
| | VH PDGFR | | | VL PDGFR | |
| | VH EpCAM | | | VL EpCAM | |
| | VH CD14 | | | VL CD14 | |
| | VH CD52 | | | VL CD52 | |
| | VH PD-L1 | | | VL PD-L1 | |
| | VH PD-1 | | | VL PD-1 | |
| | VH TIM3 | | | VL TIM3 | |
| | VH BTLA | | | VL BTLA | |
| | VH LAG3 | | | VL LAG3 | |
| | VH CTLA4 | | | VL CTLA4 | |
| | VH CD19 | | | VL CD19 | |
| | VH CD20 | | | VL CD20 | |
| | VH CD22 | | | VL CD22 | |
| | VH CD47 | | | VL CD47 | |
| | VH CD123 | | | VL CD123 | |
| | VH GD-2 | | | VL GD-2 | |
| | VH ICAM1 | | | VL ICAM1 | |
| | VH ICAM2 | | | VL ICAM2 | |
| | VH ICAM3 | | | VL ICAM3 | |
| | VH ICAM4 | | | VL ICAM4 | |
| | VH VCAM | | | VL VCAM | |
| | VH FAPa | | | VL FAPa | |
| | VH 5T4 | | | VL 5T4 | |
| | VH Trop2 | | | VL Trop2 | |
| | VH EDB-FN | | | VL EDB-FN | |
| | VH TGFb | | | VL TGFb | |
| | VH Trap | | | VL Trap | |
| | VH MAdCAM | | | VL MAdCAM | |
| | VH b7 integrin | | | VL b7 integrin | |
| | VH a4b7 | | | VL a4b7 | |
| | VH a4 | | | VL a4 | |
| | VH of anyone | | | VL of anyone | |
| | of SR² | | | of SR² | |
| | VH BCMA | | | VL BCMA | |
| | VH PSA | | | VL PSA | |
| | VH PSMA | | | VL PSMA | |
| | VH CEA | | | VL CEA | |
| | VH GPC3 | | | VL GPC3 | |

¹R1, X1, X2, and R2 may be combined with an IL-10 monomer selected wild-type human IL10, EBV IL10, CMV IL10, mouse IL10, high affinity IL10 (known as DV07), low affinity IL10 (known as DV06) having SEQ ID Nos: 31, 33, 35, 37, 39, and 41, respectively. P40 and P35 having sequences of SEQ ID Nos: 18 and 17, respectively.

TABLE 2b
(Embodiments of DK12¹⁰ Combination 2)

| (R¹) | (X¹) | (Z¹) | (Z²) | (X²) | (R²) |
|---|---|---|---|---|---|
| P40 | VL EGFR | IL10 | IL10 | VH EGFR | P35 |
| | VL HER2 | monomer¹ | monomer¹ | VH HER2 | |
| | VL HER3 | | | VH HER3 | |
| | VL VEGFR2 | | | VH VEGFR2 | |
| | VL VEGFR1 | | | VH VEGFR1 | |
| | VL PDGFR | | | VH PDGFR | |
| | VL EpCAM | | | VH EpCAM | |
| | VL CD14 | | | VH CD14 | |
| | VL CD52 | | | VH CD52 | |
| | VL PD-L1 | | | VH PD-L1 | |
| | VL PD-1 | | | VH PD-1 | |
| | VL TIM3 | | | VH TIM3 | |
| | VL BTLA | | | VH BTLA | |
| | VL LAG3 | | | VH LAG3 | |
| | VL CTLA4 | | | VH CTLA4 | |
| | VL CD19 | | | VH CD19 | |
| | VL CD20 | | | VH CD20 | |
| | VL CD22 | | | VH CD22 | |
| | VL CD47 | | | VH CD47 | |
| | VL CD123 | | | VH CD123 | |
| | VL GD-2 | | | VH GD-2 | |
| | VL ICAM1 | | | VH ICAM1 | |
| | VL ICAM2 | | | VH ICAM2 | |
| | VL ICAM3 | | | VH ICAM3 | |
| | VL ICAM4 | | | VH ICAM4 | |
| | VL VCAM | | | VH VCAM | |
| | VL FAPa | | | VH FAPa | |
| | VL 5T4 | | | VH 5T4 | |
| | VL Trop2 | | | VH Trop2 | |
| | VL EDB-FN | | | VH EDB-FN | |
| | VL TGFb | | | VH TGFb | |
| | VL Trap | | | VH Trap | |
| | VL MAdCAM | | | VH MAdCAM | |
| | VL b7 integrin | | | VH b7 integrin | |
| | VL a4b7 | | | VH a4b7 | |
| | VL a4 | | | VH a4 | |
| | VL of anyone | | | VH of anyone | |
| | of SR² | | | of SR² | |
| | VL BCMA | | | VH BCMA | |
| | VL PSA | | | VH PSA | |
| | VL PSMA | | | VH PSMA | |
| | VL CEA | | | VH CEA | |
| | VL GPC3 | | | VH GPC3 | |

²SR-A1; SR-A3; SR-A4; SR-A5; SR-A6; SR-B; dSR-C1; SR-D1; SR-E1; SR-F1; SR-F2; SR-G; SR-H1; SR-H2; SR-I1; or SR-J1.

TABLE 2c
(Embodiments of DK12¹⁰ Combination 3)

| (R¹) | (X¹) | (Z¹) | (Z²) | (X²) | (R²) |
|---|---|---|---|---|---|
| P35 | VH EGFR | IL10 | IL10 | VL EGFR | P40 |
| | VH HER2 | monomer¹ | monomer¹ | VL HER2 | |
| | VH HER3 | | | VL HER3 | |
| | VH VEGFR2 | | | VL VEGFR2 | |
| | VH VEGFR1 | | | VL VEGFR1 | |
| | VH PDGFR | | | VL PDGFR | |
| | VH EpCAM | | | VL EpCAM | |
| | VH CD14 | | | VL CD14 | |
| | VH CD52 | | | VL CD52 | |
| | VH PD-L1 | | | VL PD-L1 | |
| | VH PD-1 | | | VL PD-1 | |
| | VH TIM3 | | | VL TIM3 | |
| | VH BTLA | | | VL BTLA | |
| | VH LAG3 | | | VL LAG3 | |
| | VH CTLA4 | | | VL CTLA4 | |
| | VH CD19 | | | VL CD19 | |
| | VH CD20 | | | VL CD20 | |
| | VH CD22 | | | VL CD22 | |
| | VH CD47 | | | VL CD47 | |
| | VH CD123 | | | VL CD123 | |
| | VH GD-2 | | | VL GD-2 | |
| | VH ICAM1 | | | VL ICAM1 | |
| | VH ICAM2 | | | VL ICAM2 | |
| | VH ICAM3 | | | VL ICAM3 | |
| | VH ICAM4 | | | VL ICAM4 | |
| | VH VCAM | | | VL VCAM | |
| | VH FAPa | | | VL FAPa | |
| | VH 5T4 | | | VL 5T4 | |
| | VH Trop2 | | | VL Trop2 | |
| | VH EDB-FN | | | VL EDB-FN | |
| | VH TGFb | | | VL TGFb | |
| | VH Trap | | | VL Trap | |
| | VH MAdCAM | | | VL MAdCAM | |
| | VH b7 integrin | | | VL b7 integrin | |
| | VH a4b7 | | | VL a4b7 | |
| | VH a4 | | | VL a4 | |
| | VH of anyone | | | VL of anyone | |
| | of SR² | | | of SR² | |
| | VH BCMA | | | VL BCMA | |
| | VH PSA | | | VL PSA | |
| | VH PSMA | | | VL PSMA | |
| | VH CEA | | | VL CEA | |
| | VH GPC3 | | | VL GPC3 | |

TABLE 2d
(Embodiments of DK12¹⁰ Combination 4)

| (R¹) | (X¹) | (Z¹) | (Z²) | (X²) | (R²) |
|---|---|---|---|---|---|
| P35 | VL EGFR | IL10 | IL10 | VH EGFR | P40 |
| | VL HER2 | monomer¹ | monomer¹ | VH HER2 | |
| | VL HER3 | | | VH HER3 | |
| | VL VEGFR2 | | | VH VEGFR2 | |
| | VL VEGFR1 | | | VH VEGFR1 | |
| | VL PDGFR | | | VH PDGFR | |
| | VL EpCAM | | | VH EpCAM | |
| | VL CD14 | | | VH CD14 | |
| | VL CD52 | | | VH CD52 | |
| | VL PD-L1 | | | VH PD-L1 | |
| | VL PD-1 | | | VH PD-1 | |
| | VL TIM3 | | | VH TIM3 | |
| | VL BTLA | | | VH BTLA | |
| | VL LAG3 | | | VH LAG3 | |
| | VL CTLA4 | | | VH CTLA4 | |
| | VL CD19 | | | VH CD19 | |
| | VL CD20 | | | VH CD20 | |
| | VL CD22 | | | VH CD22 | |
| | VL CD47 | | | VH CD47 | |
| | VL CD123 | | | VH CD123 | |
| | VL GD-2 | | | VH GD-2 | |
| | VL ICAM1 | | | VH ICAM1 | |
| | VL ICAM2 | | | VH ICAM2 | |
| | VL ICAM3 | | | VH ICAM3 | |
| | VL ICAM4 | | | VH ICAM4 | |
| | VL VCAM | | | VH VCAM | |
| | VL FAPa | | | VH FAPa | |
| | VL 5T4 | | | VH 5T4 | |
| | VL Trop2 | | | VH Trop2 | |
| | VL EDB-FN | | | VH EDB-FN | |
| | VL TGFb | | | VH TGFb | |
| | VL Trap | | | VH Trap | |
| | VL MAdCAM | | | VH MAdCAM | |
| | VL b7 integrin | | | VH b7 integrin | |
| | VL a4b7 | | | VH a4b7 | |
| | VL a4 | | | VH a4 | |
| | VL of anyone | | | VH of anyone | |
| | of SR² | | | of SR² | |
| | VL BCMA | | | VH BCMA | |
| | VL PSA | | | VH PSA | |
| | VL PSMA | | | VH PSMA | |
| | VL CEA | | | VH CEA | |

The IL-12 subunits, P40 and P35, as noted in Tables 2a-2d above, may be selected from wild type, deglycosylated, or aglycosylated forms of IL-12. In addition, the IL-12 may be derived from human IL-12 or mouse IL-12 or any variant of IL-12 that retains, enhances, or decreases the function of IL-12, when compared to wild type IL-12. The IL-10 monomers listed in Table 2a-2d above, may be selected from human IL-10, EBV IL-10, CMV IL-10, high affinity variant forms of IL-10 (such as DV07, SEQ ID 41), or low affinity variant forms of IL-10 (such as DV06, SEQ ID 39). Moreover, IL-10 monomers may be any variant of IL-10 that retains, enhances, or decreases the function of IL-10, when compared to wild type IL-10.

In another one embodiment, Tables 3a-3d are different combinations of a dual cytokine fusion protein comprising IL-27 and IL-10 as represented by Formula IV.

TABLE 3a
(Embodiments of DK27¹⁰ Combination 1)

| (R¹) | (X¹) | (Z¹) | (Z²) | (X²) | (R²) |
|---|---|---|---|---|---|
| P28 | VH EGFR | IL-10 | IL-10 | VL EGFR | EBI3 |
| | VH HER2 | Monomer³ | Monomer³ | VL HER2 | |
| | VH HER3 | | | VL HER3 | |
| | VH VEGFR2 | | | VL VEGFR2 | |
| | VH VEGFR1 | | | VL VEGFR1 | |
| | VH PDGFR | | | VL PDGFR | |
| | VH EpCAM | | | VL EpCAM | |
| | VH CD14 | | | VL CD14 | |
| | VH CD52 | | | VL CD52 | |
| | VH PD-L1 | | | VL PD-L1 | |
| | VH PD-1 | | | VL PD-1 | |
| | VH TIM3 | | | VL TIM3 | |
| | VH BTLA | | | VL BTLA | |
| | VH LAG3 | | | VL LAG3 | |
| | VH CTLA4 | | | VL CTLA4 | |
| | VH CD19 | | | VL CD19 | |
| | VH CD20 | | | VL CD20 | |
| | VH CD22 | | | VL CD22 | |
| | VH CD47 | | | VL CD47 | |
| | VH CD123 | | | VL CD123 | |
| | VH GD-2 | | | VL GD-2 | |
| | VH ICAM1 | | | VL ICAM1 | |
| | VH ICAM2 | | | VL ICAM2 | |
| | VH ICAM3 | | | VL ICAM3 | |
| | VH ICAM4 | | | VL ICAM4 | |
| | VH VCAM | | | VL VCAM | |
| | VH FAPa | | | VL FAPa | |
| | VH 5T4 | | | VL 5T4 | |
| | VH Trop2 | | | VL Trop2 | |
| | VH EDB-FN | | | VL EDB-FN | |
| | VH TGFb | | | VL TGFb | |
| | VH Trap | | | VL Trap | |
| | VH MAdCAM | | | VL MAdCAM | |
| | VH b7 integrin | | | VL b7 integrin | |
| | VH a4b7 | | | VL a4b7 | |
| | VH a4 | | | VL a4 | |
| | VH of anyone | | | VL of anyone | |
| | of SR² | | | of SR² | |
| | VH BCMA | | | VL BCMA | |
| | VH PSA | | | VL PSA | |
| | VH PSMA | | | VL PSMA | |
| | VH CEA | | | VL CEA | |
| | VH GPC3 | | | VL GPC3 | |

³R1, X1, X2, and R2 may be combined with an IL-10 monomer selected wild-type human IL10, EBV IL10, CMV IL10, mouse IL10, high affinity IL10 (known as DV07), low affinity IL10 (known as DV06) having SEQ ID Nos: 31, 33, 35, 37, 39, and 41, respectively. P40 and P35 having sequences of SEQ ID Nos: 18 and 17, respectively

TABLE 3b
(Embodiments of DK27¹⁰ Combination 2)

| (R¹) | (X¹) | (Z¹) | (Z²) | (X²) | (R²) |
|---|---|---|---|---|---|
| P28 | VL EGFR | IL-10 | IL-10 | VH EGFR | EBI3 |
| | VL HER2 | Monomer³ | Monomer³ | VH HER2 | |
| | VL HER3 | | | VH HER3 | |
| | VL VEGFR2 | | | VH VEGFR2 | |
| | VL VEGFR1 | | | VH VEGFR1 | |
| | VL PDGFR | | | VH PDGFR | |
| | VL EpCAM | | | VH EpCAM | |
| | VL CD14 | | | VH CD14 | |
| | VL CD52 | | | VH CD52 | |
| | VL PD-L1 | | | VH PD-L1 | |
| | VL PD-1 | | | VH PD-1 | |
| | VL TIM3 | | | VH TIM3 | |
| | VL BTLA | | | VH BTLA | |
| | VL LAG3 | | | VH LAG3 | |
| | VL CTLA4 | | | VH CTLA4 | |
| | VL CD19 | | | VH CD19 | |
| | VL CD20 | | | VH CD20 | |
| | VL CD22 | | | VH CD22 | |
| | VL CD47 | | | VH CD47 | |
| | VL CD123 | | | VH CD123 | |
| | VL GD-2 | | | VH GD-2 | |
| | VL ICAM1 | | | VH ICAM1 | |
| | VL ICAM2 | | | VH ICAM2 | |
| | VL ICAM3 | | | VH ICAM3 | |
| | VL ICAM4 | | | VH ICAM4 | |
| | VL VCAM | | | VH VCAM | |
| | VL FAPa | | | VH FAPa | |
| | VL 5T4 | | | VH 5T4 | |
| | VL Trop2 | | | VH Trop2 | |
| | VL EDB-FN | | | VH EDB-FN | |
| | VL TGFb | | | VH TGFb | |
| | VL Trap | | | VH Trap | |
| | VL MAdCAM | | | VH MAdCAM | |
| | VL b7 integrin | | | VH b7 integrin | |
| | VL a4b7 | | | VH a4b7 | |
| | VL a4 | | | VH a4 | |
| | VL of anyone | | | VH of anyone | |
| | of SR² | | | of SR² | |
| | VL BCMA | | | VH BCMA | |
| | VL PSA | | | VH PSA | |
| | VL PSMA | | | VH PSMA | |
| | VL CEA | | | VH CEA | |
| | VL GPC3 | | | VH GPC3 | |

TABLE 3c
(Embodiments of DK27¹⁰ Combination 3)

| (R¹) | (X¹) | (Z¹) | (Z²) | (X²) | (R²) |
|---|---|---|---|---|---|
| EBI3 | VH EGFR | IL-10 | IL-10 | VL EGFR | P28 |
| | VH HER2 | Monomer³ | Monomer³ | VL HER2 | |
| | VH HER3 | | | VL HER3 | |
| | VH VEGFR2 | | | VL VEGFR2 | |
| | VH VEGFR1 | | | VL VEGFR1 | |
| | VH PDGFR | | | VL PDGFR | |
| | VH EpCAM | | | VL EpCAM | |
| | VH CD14 | | | VL CD14 | |
| | VH CD52 | | | VL CD52 | |
| | VH PD-L1 | | | VL PD-L1 | |
| | VH PD-1 | | | VL PD-1 | |
| | VH TIM3 | | | VL TIM3 | |
| | VH BTLA | | | VL BTLA | |
| | VH LAG3 | | | VL LAG3 | |
| | VH CTLA4 | | | VL CTLA4 | |
| | VH CD19 | | | VL CD19 | |
| | VH CD20 | | | VL CD20 | |
| | VH CD22 | | | VL CD22 | |
| | VH CD47 | | | VL CD47 | |
| | VH CD123 | | | VL CD123 | |
| | VH GD-2 | | | VL GD-2 | |
| | VH ICAM1 | | | VL ICAM1 | |
| | VH ICAM2 | | | VL ICAM2 | |
| | VH ICAM3 | | | VL ICAM3 | |
| | VH ICAM4 | | | VL ICAM4 | |
| | VH VCAM | | | VL VCAM | |
| | VH FAPa | | | VL FAPa | |
| | VH 5T4 | | | VL 5T4 | |
| | VH Trop2 | | | VL Trop2 | |
| | VH EDB-FN | | | VL EDB-FN | |
| | VH TGFb | | | VL TGFb | |
| | VH Trap | | | VL Trap | |
| | VH MAdCAM | | | VL MAdCAM | |
| | VH b7 integrin | | | VL b7 integrin | |
| | VH a4b7 | | | VL a4b7 | |
| | VH a4 | | | VL a4 | |
| | VH of anyone | | | VL of anyone | |
| | of SR² | | | of SR² | |
| | VH BCMA | | | VL BCMA | |
| | VH PSA | | | VL PSA | |
| | VH PSMA | | | VL PSMA | |
| | VH CEA | | | VL CEA | |
| | VH GPC3 | | | VL GPC3 | |

TABLE 3d
(Embodiments of DK27¹⁰ Combination 4)

| (R¹) | (X¹) | (Z¹) | (Z²) | (X²) | (R²) |
|---|---|---|---|---|---|
| EBI3 | VL EGFR | IL-10 | IL-10 | VH EGFR | P28 |
| | VL HER2 | Monomer³ | Monomer³ | VH HER2 | |
| | VL HER3 | | | VH HER3 | |
| | VL VEGFR2 | | | VH VEGFR2 | |
| | VL VEGFR1 | | | VH VEGFR1 | |
| | VL PDGFR | | | VH PDGFR | |
| | VL EpCAM | | | VH EpCAM | |
| | VL CD14 | | | VH CD14 | |
| | VL CD52 | | | VH CD52 | |
| | VL PD-L1 | | | VH PD-L1 | |
| | VL PD-1 | | | VH PD-1 | |
| | VL TIM3 | | | VH TIM3 | |
| | VL BTLA | | | VH BTLA | |
| | VL LAG3 | | | VH LAG3 | |
| | VL CTLA4 | | | VH CTLA4 | |
| | VL CD19 | | | VH CD19 | |
| | VL CD20 | | | VH CD20 | |
| | VL CD22 | | | VH CD22 | |
| | VL CD47 | | | VH CD47 | |
| | VL CD123 | | | VH CD123 | |
| | VL GD-2 | | | VH GD-2 | |
| | VL ICAM1 | | | VH ICAM1 | |
| | VL ICAM2 | | | VH ICAM2 | |
| | VL ICAM3 | | | VH ICAM3 | |
| | VL ICAM4 | | | VH ICAM4 | |
| | VL VCAM | | | VH VCAM | |
| | VL FAPa | | | VH FAPa | |
| | VL 5T4 | | | VH 5T4 | |
| | VL Trop2 | | | VH Trop2 | |
| | VL EDB-FN | | | VH EDB-FN | |
| | VL TGFb | | | VH TGFb | |
| | VL Trap | | | VH Trap | |
| | VL MAdCAM | | | VH MAdCAM | |
| | VL b7 integrin | | | VH b7 integrin | |
| | VL a4b7 | | | VH a4b7 | |
| | VL a4 | | | VH a4 | |
| | VL of anyone | | | VH of anyone | |
| | of SR² | | | of SR² | |
| | VL BCMA | | | VH BCMA | |
| | VL PSA | | | VH PSA | |
| | VL PSMA | | | VH PSMA | |
| | VL CEA | | | VH CEA | |
| | VL GPC3 | | | VH GPC3 | |

The IL-27 subunits, P28 and EBI3, as noted in Tables 3a-3d above, may be selected from wild type, deglycosylated, or aglycosylated forms of IL-27. In addition, the IL-27 may be derived from human IL-27 or mouse IL-27 or any variant of IL-27 that retains, enhances, or decreases the function of IL-27, when compared to wild type IL-27. The IL-10 monomers listed in Table 2a-2d above, may be selected from human IL-10, EBV IL-10, CMV IL-10, high affinity variant forms of IL-10 (such as DV07, SEQ ID 41), or low affinity variant forms of IL-10 (such as DV06, SEQ ID 39). Moreover, IL-10 monomers may be any variant of IL-10 that retains, enhances, or decreases the function of IL-10, when compared to wild type IL-10.

In another embodiment, the target specificity of the antibody variable chains or VH and VL pair or the 6 CDRs of the VH and VL pair may include, but not limited to those targeting proteins, cellular receptors, and/or tumor associated antigens. In another embodiment, the CDR regions from any VH and VL pair may be engrafted into the dual cytokine scaffolding system described above (schematically represented by FIG. 1-5). In yet another embodiment, the variable regions or VH and VL pair or the 6 CDRs of the VH and VL pair are obtained from antibodies that target antigens associated with various diseases (e.g., cancer) or those that are not typically found or rarely found in the serum of a healthy subject, for example variable regions from antibodies directed to EGFR, PDGFR, VEGFR1, VEGFR2, Her2Neu, FGFR, GPC3, or other tumor associated antigens, MAdCAM, ICAM, VCAM, CD14 or other inflammation associated cell surface proteins, HIV and/or Ebola. Thus, in one embodiment, the variable regions are obtained or derived from anti-EGFR, anti-MAdCAM, anti-HIV (Chan et al, J. Virol., 2018, 92(18):e006411-19), anti-ICAM, anti-VCAM, anti-CD14, or anti-Ebola (US Published Application 2018/0180614, incorporated by reference in its entirety, especially mAbs described in Tables 2, 3, and 4) antibodies, for example. In another embodiment, the variable regions are obtained or derived from antibodies capable of enriching the concentration of cytokines, such as IL-12 or IL-27 in combination with IFN-alpha, IL-28, or IL-29, to a specific target area so as to enable IL-12 and IL-27 to elicit its biological effect more effectively. Such an antibody might include those that target overexpressed or upregulated receptors or antigens in certain diseased regions or those that are specifically expressed in certain impacted areas. For example, the variable regions or CDRs might be obtained from antibodies specific for EGFR; CD14; CD52; various immune check point targets, such as but not limited to PD-L1, PD-1, TIM3, BTLA, LAG3 or CTLA4; CD19; CD20; CD22; CD47; CD123; GD-2; VEGFR1; VEGFR2; HER2; PDGFR; EpCAM; ICAM (ICAM-1, -2, -3, -4, -5), VCAM, FAPα; 5T4; Trop2; EDB-FN; TGFβ Trap; MAdCAM, β7 integrin subunit; α4β7 integrin; α4 integrin; BCMA; PSA; PSMA; CEA; GPC3; SR-A1; SR-A3; SR-A4; SR-A5; SR-A6; SR-B; dSR-C1; SR-D1; SR-E1; SR-F1; SR-F2; SR-G; SR-H1; SR-H2; SR-I1; or SR-J1 to name a few.

The dual cytokine fusion protein or dual cytokine fusion protein complex may also have an antigen targeting functionality. The dual cytokine fusion protein or dual cytokine fusion protein complex will comprise a VH and VL pair that is able to associate together to form an antigen binding site or ABS. The variable regions may be further modified (e.g., by addition, subtraction, or substitution) by altering one or more amino acids that reduce antigenicity in a subject. Other modifications to the variable region may include amino acids substitutions, deletions, or additions that are found outside of the 6 CDR regions of the VH and VL regions and serve to increase stability and expression of the VH and VL regions of the scFv. A person of skill in the art would be capable of determining other modifications that stabilize the scFv and/or to optimize the sequence for purposes of expression.

The VH and VL pair form a scaffolding onto which CDR regions obtained for a plurality of antibodies may be substituted or engrafted. Such antibody CDR regions include those antibodies known and described above. The CDR regions in the above described VH and VL scaffolding will include the following number of amino acid positions available for CDR engraftment/insertion:

| | | |
|---|---|---|
| Heavy chain CDR1 | 3-7 | amino acids |
| Heavy chain CDR2 | 7-11 | amino acids |
| Heavy chain CDR3 | 7-11 | amino acids |
| Light chain CDR1 | 9-14 | amino acids |
| Light chain CDR2 | 5-9 | amino acids |
| Light chain CDR3 | 7-11 | amino acids |

In a preferred embodiment, the dual cytokine fusion protein comprising a first multi-subunit cytokine (e.g., IL-12 or IL-27) will include a VH and VL pair is derived from a human anti-ebola antibody (US Published Application 2018/0180614, incorporated by reference in its entirety, especially mAbs described in Tables 2, 3, and 4) whereby the 6 CDR regions from the human anti-ebola antibody are removed and engrafted with a VH and VL pair of a specific targeting antibody, such as but not limited to antibodies that target EGFR; CD14; CD52; various immune check point targets, such as but not limited to PD-L1, PD-1, TIM3, BTLA, LAG3 or CTLA4; CD19; CD20; CD22; CD47; CD123; GD-2; VEGFR1; VEGFR2; HER2; PDGFR; EpCAM; ICAM (ICAM-1, -2, -3, -4, -5), VCAM, FAPα; 5T4; Trop2; EDB-FN; TGFβ Trap; MAdCAM, β7 integrin subunit; α4β7 integrin; α4 integrin; BCMA; PSA; PSMA; CEA; GPC3; SR-A1; SR-A3; SR-A4; SR-A5; SR-A6; SR-B; dSR-C1; SR-D1; SR-E1; SR-F1; SR-F2; SR-G; SR-H1; SR-H2; SR-I1; or SR-J1. In an embodiment, the 6 human anti-ebola CDR regions are substituted with 6 CDR regions from anti-EGFR, anti-MAdCAM, anti-VEGFR1, anti-VEGFR2, anti-PDGFR, or anti-CD14. In a preferred embodiment, a second cytokine, such as but not limited to IL-28, IL-29, IFNα, is linked in the hinge region between the VH and VL of the scFv obtained from a human anti-ebola antibody. The aforementioned engraftment strategy may also be applied to the dual cytokine fusion protein comprising two multi-subunit cytokines as represented by Formula IV and Va and Vb recited above.

In yet another embodiment, the second cytokine, is fused between the VH and VL of a scFv, as depicted in FIG. 1-5. The second cytokine is conjugated between the VH or VL region such that the second cytokine retains its functional properties. In one embodiment, the second cytokine is different from the first multi-subunit cytokine (e.g., IL-12 or IL-27). In one embodiment, the second cytokine is IL-6, IL-4, IL-1, IL-2, IL-3, IL-5, IL-7, IL-8, IL-9, IL-15, IL-21, IL-26, IL-27, IL-28, IL-29, GM-CSF, G-CSF, interferons-α, -β, -γ, TGF-β, or tumor necrosis factors-α, -β, basic FGF, EGF, PDGF, IL-4, IL-11, or IL-13. In a preferred embodiment, the second cytokine in the dual cytokine fusion protein comprising IL-28, IL-29 or IFN-alpha.

In some embodiments, the second cytokine may be another multi-subunit cytokine, such as IL-10. In this case, the second cytokine will also be fused between the VH and VL of the scaffolding system (see FIGS. 7 and 8 for representative structures). Formulae IV, Va and Vb, described above, serve to describe these types of fusion proteins.

In still other embodiments, the dual cytokine fusion protein comprising a first multi-subunit cytokine (e.g., IL-12 or IL-27) incorporates linkers. A person of skill in the art knows that linkers or spacers are used to achieve proper spatial configuration of the various fusion protein parts and therefore may select the appropriate linker to use in the formation of the dual cytokine fusion protein comprising the first multi-subunit cytokine (e.g., IL-12 or IL-27). In a more preferred embodiment, the linker or spacer may be a random amino acid sequence SEQ ID Nos.: 43, 44, 45, 46, 47, and 48. Any of the above combinations in Tables 2a-2d or 3a-3d may be combined with linker (“Lₐ”) and “(L_{b}”) selected from (GGGGS)₃, (GGGGS)₄, or (GGGGS)₅ having SEQ ID Nos: 46, 45, and 47 respectively, and a linker (“L_{c}”) of GGGSGGG or (GGGGS)₃ corresponding to SEQ ID Nos: 43 and 46, respectively. In one preferred embodiment, Formula IV may include a combination of linkers as follows in Table 4.

TABLE 4
Possible Linker Combinations in Formula IV

| | | | | IL-10 | | IL-10 | | | | |
|---|---|---|---|---|---|---|---|---|---|---|
| IL-12 | Lₐ | | | monomer | | monomer | | | | IL-12 |
| p40 | a | VH | L_{b} | 1 | L_{c} | 2 | L_{b} | VL | Lₐ | p35 |
| X | GGGGS | X | GGGGS | X | GGGSGGG | X | GGGGS | X | GGGGS | X |
| | x 5 | | x 5 | | | | x 5 | | x 5 | |
| X | GGGGS | X | GGGGS | X | GGGSGGG | X | GGGGS | X | GGGGS | X |
| | x 4 | | x 4 | | | | x 4 | | x 4 | |
| X | GGGGS | X | GGGGS | X | GGGSGGG | X | GGGGS | X | GGGGS | X |
| | x 3 | | x 3 | | | | x 3 | | x 3 | |
| X | GGGGS | X | GGGGS | X | GGGSGGG | X | GGGGS | X | GGGGS | X |
| | x 5 | | x 5 | | | | x 5 | | x 5 | |
| X | GGGGS | X | GGGGS | X | GGGSGGG | X | GGGGS | X | GGGGS | X |
| | x 5 | | x 4 | | | | x 4 | | x 4 | |
| X | GGGGS | X | GGGGS | X | GGGSGGG | X | GGGGS | X | GGGGS | X |
| | x 5 | | x 3 | | | | x 3 | | x 3 | |
| X | GGGGS | X | GGGGS | X | GGGSGGG | X | GGGGS | X | GGGGS | X |
| | x 4 | | x 5 | | | | x 5 | | x 5 | |
| X | GGGGS | X | GGGGS | X | GGGSGGG | X | GGGGS | X | GGGGS | X |
| | x 4 | | x 4 | | | | x 4 | | x 4 | |
| X | GGGGS | X | GGGGS | X | GGGSGGG | X | GGGGS | X | GGGGS | X |
| | x 4 | | x 3 | | | | x 3 | | x 3 | |
| X | GGGGS | X | GGGGS | X | GGGSGGG | X | GGGGS | X | GGGGS | X |
| | x 3 | | x 5 | | | | x 5 | | x 5 | |
| X | GGGGS | X | GGGGS | X | GGGSGGG | X | GGGGS | X | GGGGS | X |
| | x 3 | | x 4 | | | | x 4 | | x 4 | |
| X | GGGGS | X | GGGGS | X | GGGSGGG | X | GGGGS | X | GGGGS | X |
| | x 3 | | x 3 | | | | x 3 | | x 3 | |
| X | GGGGS | X | GGGGS | X | GGGSGGG | X | GGGGS | X | GGGGS | X |
| | x 5 | | x 5 | | | | x 5 | | x 5 | |
| X | GGGGS | X | GGGGS | X | GGGSGGG | X | GGGGS | X | GGGGS | X |
| | x 4 | | x 5 | | | | x 4 | | x 4 | |
| X | GGGGS | X | GGGGS | X | GGGSGGG | X | GGGGS | X | GGGGS | X |
| | x 3 | | x 5 | | | | x 3 | | x 3 | |
| X | GGGGS | X | GGGGS | X | GGGSGGG | X | GGGGS | X | GGGGS | X |
| | x 5 | | x 4 | | | | x 5 | | x 5 | |
| X | GGGGS | X | GGGGS | X | GGGSGGG | X | GGGGS | X | GGGGS | X |
| | x 4 | | x 4 | | | | x 4 | | x 4 | |
| X | GGGGS | X | GGGGS | X | GGGSGGG | X | GGGGS | X | GGGGS | X |
| | x 3 | | x 4 | | | | x 3 | | x 3 | |
| X | GGGGS | X | GGGGS | X | GGGSGGG | X | GGGGS | X | GGGGS | X |
| | x 5 | | x 3 | | | | x 5 | | x 5 | |
| X | GGGGS | X | GGGGS | X | GGGSGGG | X | GGGGS | X | GGGGS | X |
| | x 4 | | x 3 | | | | x 4 | | x 4 | |
| X | GGGGS | X | GGGGS | X | GGGSGGG | X | GGGGS | X | GGGGS | X |
| | x 3 | | x 3 | | | | x 3 | | x 3 | |
| X | GGGGS | X | GGGGS | X | GGGSGGG | X | GGGGS | X | GGGGS | X |
| | x 5 | | x 5 | | | | x 5 | | x 5 | |
| X | GGGGS | X | GGGGS | X | GGGSGGG | X | GGGGS | X | GGGGS | X |
| | x 4 | | x 4 | | | | x 5 | | x 4 | |
| X | GGGGS | X | GGGGS | x | GGGSGGG | X | GGGGS | X | GGGGS | X |
| | x 3 | | x 3 | | | | x 5 | | x 3 | |
| X | GGGGS | X | GGGGS | X | GGGSGGG | X | GGGGS | X | GGGGS | X |
| | x 5 | | x 5 | | | | x 4 | | x 5 | |
| X | GGGGS | X | GGGGS | X | GGGSGGG | X | GGGGS | X | GGGGS | X |
| | x 4 | | x 4 | | | | x 4 | | x 4 | |
| X | GGGGS | X | GGGGS | X | GGGSGGG | X | GGGGS | X | GGGGS | X |
| | x 3 | | x 3 | | | | x 4 | | x 3 | |
| X | GGGGS | X | GGGGS | X | GGGSGGG | X | GGGGS | X | GGGGS | X |
| | x 5 | | x 5 | | | | x 3 | | x 5 | |
| X | GGGGS | X | GGGGS | X | GGGSGGG | X | GGGGS | X | GGGGS | X |
| | x 4 | | x 4 | | | | x 3 | | x 4 | |
| X | GGGGS | X | GGGGS | X | GGGSGGG | X | GGGGS | X | GGGGS | X |
| | x 3 | | x 3 | | | | x 3 | | x 3 | |
| X | GGGGS | X | GGGGS | X | GGGSGGG | X | GGGGS | X | GGGGS | X |
| | x 5 | | x 5 | | | | x 5 | | x 5 | |
| X | GGGGS | X | GGGGS | X | GGGSGGG | X | GGGGS | X | GGGGS | X |
| | x 4 | | x 4 | | | | x 5 | | x 4 | |
| X | GGGGS | X | GGGGS | X | GGGSGGG | X | GGGGS | X | GGGGS | X |
| | x 3 | | x 3 | | | | x 5 | | x 3 | |
| X | GGGGS | X | GGGGS | X | GGGSGGG | X | GGGGS | X | GGGGS | X |
| | x 5 | | x 5 | | | | x 3 | | x 5 | |
| X | GGGGS | X | GGGGS | X | GGGSGGG | X | GGGGS | X | GGGGS | X |
| | x 4 | | x 4 | | | | x 3 | | x 4 | |
| X | GGGGS | X | GGGGS | X | GGGSGGG | X | GGGGS | X | GGGGS | X |
| | x 3 | | x 3 | | | | x 3 | | x 3 | |
| X | GGGGS | X | GGGGS | X | GGGSGGG | X | GGGGS | X | GGGGS | X |
| | x 5 | | x 5 | | | | x 5 | | x 5 | |
| X | GGGGS | X | GGGGS | X | GGGSGGG | X | GGGGS | X | GGGGS | X |
| | x 4 | | x 4 | | | | x 4 | | x 5 | |
| X | GGGGS | X | GGGGS | X | GGGSGGG | X | GGGGS | X | GGGGS | X |
| | x 3 | | x 3 | | | | x 3 | | x 5 | |
| X | GGGGS | X | GGGGS | X | GGGGS | X | GGGGS | X | GGGGS | X |
| | x 5 | | x 5 | | x 3 | | x 5 | | x 5 | |
| X | GGGGS | X | GGGGS | X | GGGGS | X | GGGGS | X | GGGGS | X |
| | x 4 | | x 4 | | x 3 | | x 4 | | x 4 | |
| X | GGGGS | X | GGGGS | X | GGGGS | X | GGGGS | X | GGGGS | X |
| | x 3 | | x 3 | | x 3 | | x 3 | | x 3 | |
| X | GGGGS | X | GGGGS | X | GGGGS | X | GGGGS | X | GGGGS | X |
| | x 5 | | x 5 | | x 3 | | x 5 | | x 5 | |
| X | GGGGS | X | GGGGS | X | GGGGS | X | GGGGS | X | GGGGS | X |
| | x 5 | | x 4 | | x 3 | | x 4 | | x 4 | |
| X | GGGGS | X | GGGGS | X | GGGGS | X | GGGGS | X | GGGGS | X |
| | x 5 | | x 3 | | x 3 | | x 3 | | x 3 | |
| X | GGGGS | X | GGGGS | X | GGGGS | X | GGGGS | X | GGGGS | X |
| | x 4 | | x 5 | | x 3 | | x 5 | | x 5 | |
| X | GGGGS | X | GGGGS | X | GGGGS | X | GGGGS | X | GGGGS | X |
| | x 4 | | x 4 | | x 3 | | x 4 | | x 4 | |
| X | GGGGS | X | GGGGS | X | GGGGS | X | GGGGS | X | GGGGS | X |
| | x 4 | | x 3 | | x 3 | | x 3 | | x 3 | |
| X | GGGGS | X | GGGGS | X | GGGGS | X | GGGGS | X | GGGGS | X |
| | x 3 | | x 5 | | x 3 | | x 5 | | x 5 | |
| X | GGGGS | X | GGGGS | X | GGGGS | X | GGGGS | X | GGGGS | X |
| | x 3 | | x 4 | | x 3 | | x 4 | | x 4 | |
| X | GGGGS | X | GGGGS | X | GGGGS | X | GGGGS | X | GGGGS | X |
| | x 3 | | x 3 | | x 3 | | x 3 | | x 3 | |
| X | GGGGS | X | GGGGS | X | GGGGS | X | GGGGS | X | GGGGS | X |
| | x 5 | | x 5 | | x 3 | | x 5 | | x 5 | |
| X | GGGGS | X | GGGGS | X | GGGGS | X | GGGGS | X | GGGGS | X |
| | x 4 | | x 5 | | x 3 | | x 4 | | x 4 | |
| X | GGGGS | X | GGGGS | X | GGGGS | X | GGGGS | X | GGGGS | X |
| | x 3 | | x 5 | | x 3 | | x 3 | | x 3 | |
| X | GGGGS | X | GGGGS | X | GGGGS | X | GGGGS | X | GGGGS | X |
| | x 5 | | x 4 | | x 3 | | x 5 | | x 5 | |
| X | GGGGS | X | GGGGS | X | GGGGS | X | GGGGS | X | GGGGS | X |
| | x 4 | | x 4 | | x 3 | | x 4 | | x 4 | |
| X | GGGGS | X | GGGGS | X | GGGGS | X | GGGGS | X | GGGGS | X |
| | x 3 | | x 4 | | x 3 | | x 3 | | x 3 | |
| X | GGGGS | X | GGGGS | X | GGGGS | X | GGGGS | X | GGGGS | X |
| | x 5 | | x 3 | | x 3 | | x 5 | | x 5 | |
| X | GGGGS | X | GGGGS | X | GGGGS | X | GGGGS | X | GGGGS | X |
| | x 4 | | x 3 | | x 3 | | x 4 | | x 4 | |
| X | GGGGS | X | GGGGS | X | GGGGS | X | GGGGS | X | GGGGS | X |
| | x 3 | | x 3 | | x 3 | | x 3 | | x 3 | |
| X | GGGGS | X | GGGGS | X | GGGGS | X | GGGGS | X | GGGGS | X |
| | x 5 | | x 5 | | x 3 | | x 5 | | x 5 | |
| X | GGGGS | X | GGGGS | X | GGGGS | X | GGGGS | X | GGGGS | X |
| | x 4 | | x 4 | | x 3 | | x 5 | | x 4 | |
| X | GGGGS | X | GGGGS | X | GGGGS | X | GGGGS | X | GGGGS | X |
| | x 3 | | x 3 | | x 3 | | x 5 | | x 3 | |
| X | GGGGS | X | GGGGS | X | GGGGS | X | GGGGS | X | GGGGS | X |
| | x 5 | | x 5 | | x 3 | | x 4 | | x 5 | |
| X | GGGGS | X | GGGGS | X | GGGGS | X | GGGGS | X | GGGGS | X |
| | x 4 | | x 4 | | x 3 | | x 4 | | x 4 | |
| X | GGGGS | X | GGGGS | X | GGGGS | X | GGGGS | X | GGGGS | X |
| | x 3 | | x 3 | | x 3 | | x 4 | | x 3 | |
| X | GGGGS | X | GGGGS | X | GGGGS | X | GGGGS | X | GGGGS | X |
| | x 5 | | x 5 | | x 3 | | x 3 | | x 5 | |
| X | GGGGS | X | GGGGS | X | GGGGS | X | GGGGS | X | GGGGS | X |
| | x 4 | | x 4 | | x 3 | | x 3 | | x 4 | |
| X | GGGGS | X | GGGGS | X | GGGGS | X | GGGGS | X | GGGGS | X |
| | x 3 | | x 3 | | x 3 | | x 3 | | x 3 | |
| X | GGGGS | X | GGGGS | X | GGGGS | X | GGGGS | X | GGGGS | X |
| | x 5 | | x 5 | | x 3 | | x 5 | | x 5 | |
| X | GGGGS | X | GGGGS | X | GGGGS | X | GGGGS | X | GGGGS | X |
| | x 4 | | x 4 | | x 3 | | x 5 | | x 4 | |
| X | GGGGS | X | GGGGS | X | GGGGS | X | GGGGS | X | GGGGS | X |
| | x 3 | | x 3 | | x 3 | | x 5 | | x 3 | |
| X | GGGGS | X | GGGGS | X | GGGGS | X | GGGGS | X | GGGGS | X |
| | x 5 | | x 5 | | x 3 | | x 3 | | x 5 | |
| X | GGGGS | X | GGGGS | X | GGGGS | X | GGGGS | X | GGGGS | X |
| | x 4 | | x 4 | | x 3 | | x 3 | | x 4 | |
| X | GGGGS | X | GGGGS | X | GGGGS | X | GGGGS | X | GGGGS | X |
| | x 3 | | x 3 | | x 3 | | x 3 | | x 3 | |
| X | GGGGS | X | GGGGS | X | GGGGS | X | GGGGS | X | GGGGS | X |
| | x 5 | | x 5 | | x 3 | | x 5 | | x 5 | |
| x | GGGGS | X | GGGGS | X | GGGGS | X | GGGGS | X | GGGGS | X |
| | x 4 | | x 4 | | x 3 | | x 4 | | x 5 | |
| X | GGGGS | X | GGGGS | X | GGGGS | X | GGGGS | X | GGGGS | X |
| | x 3 | | x 3 | | x 3 | | x 3 | | x 5 | |

(GGGGS)₃ = SEQ ID No: 46,
(GGGGS)₄ = SEQ ID No: 45,
(GGGGS)₅ = SEQ ID No: 44

In yet another preferred embodiment, Formula IV may include a combination of preferred linkers as follows in Table 4

TABLE 5
Preferred Formula IV Linker Combinations

| Combination | “Lₐ” | “L_{b}” | “L_{c}” |
|---|---|---|---|
| 1 | (GGGGS)₃ | (GGGGS)₃ | GGGSGGG |
| 2 | (GGGGS)₃ | (GGGGS)₃ | (GGGGS)₃ |
| 3 | (GGGGS)₃ | (GGGGS)₅ | GGGSGGG |
| 4 | (GGGGS)₄ | (GGGGS)₃ | GGGSGGG |
| 5 | (GGGGS)₄ | (GGGGS)₄ | GGGSGGG |
| 6 | (GGGGS)₄ | (GGGGS)₅ | GGGSGGG |
| 7 | (GGGGS)₄ | (GGGGS)₃ | (GGGGS)₃ |
| 8 | (GGGGS)₄ | (GGGGS)₄ | (GGGGS)₃ |
| 9 | (GGGGS)₄ | (GGGGS)₅ | (GGGGS)₃ |
| 10 | (GGGGS)₅ | (GGGGS)₃ | GGGSGGG |
| 11 | (GGGGS)₅ | (GGGGS)₄ | GGGSGGG |
| 12 | (GGGGS)₅ | (GGGGS)₅ | GGGSGGG |
| 13 | (GGGGS)₅ | (GGGGS)₃ | (GGGGS)₃ |
| 14 | (GGGGS)₅ | (GGGGS)₄ | (GGGGS)₃ |
| 15 | (GGGGS)₅ | (GGGGS)₅ | (GGGGS)₃ |

(GGGGS)₃ = SEQ ID No: 46
(GGGGS)₄ = SEQ ID No: 45
(GGGGS)₅ = = SEQ ID No: 44

Preferred dual cytokine fusion proteins comprising two multi-subunit cytokines include those recited in SEQ ID Nos: 21-30.

In other aspects, the present disclosure relates to nucleic acid molecules that encode for the dual cytokine fusion protein comprising a first multi-subunit cytokine (e.g., IL-12 or IL-27) and a second cytokine. These would include those nucleic acid sequence that encode a dual cytokine fusion protein represented by formulas Ia, Ib, IIa, IIb, IIIa, IIIb, VI, Va, or Vb. One embodiment therefore includes a nucleic acid sequence that encodes a protein that shares 70% to 99% sequence homology thereof. The polynucleotide sequences that encode for the dual cytokine fusion protein comprising a first multi-subunit cytokine (e.g., IL-12 or IL-27) and a second cytokine (e.g, IL-10, IFN-alpha, IL-28, IL-29) may also include modifications that do not alter the functional properties of the described dual cytokine fusion protein. Such modifications will employ conventional recombinant DNA techniques and methods. For example, the addition or substitution of specific amino acid sequences may be introduced into a first multi-subunit cytokine (e.g., IL-12 or IL-27) sequence at the nucleic acid (DNA) level using site-directed mutagenesis methods employing synthetic oligonucleotides, which methods are also well known in the art. In a preferred embodiment, the nucleic acid molecules encoding the dual cytokine fusion protein may include insertions, deletions, or substitutions (e.g., degenerate code) that do not alter the functionality of the first multi-subunit cytokine (e.g., IL-12 or IL-27), the second cytokine, or the VH or VL regions of the scFv. The nucleotide sequences encoding the dual cytokine fusion proteins described herein may differ from the amino acid sequences due to the degeneracy of the genetic code and may be 70-99%, preferably 70%, 75%, 80%, 85%, 90%, 95%, 96%, 97%, 98%, or 99%, homologous to the aforementioned sequences.

The nucleotide sequences encoding the dual cytokine fusion proteins described herein may further comprise well known sequences that aid in, for example, the expression, production, or secretion of the proteins. Such sequences may include, for example a leader sequence, signal peptide, and/or translation initiation sites/sequence (e.g. Kozak consensus sequence). The nucleotide sequences described herein may also include one of more restriction enzyme sites that allow for insertion into various expression systems/vectors.

In another embodiment, the nucleotide sequences encoding the dual cytokine fusion protein may be used directly in gene therapy. In one embodiment, the dual cytokine fusion protein of the present application can be delivered by any method know in the art, including direct administration of the gene in a vector encoding the dual cytokine fusion protein. Gene therapy may be accomplished using plasmid DNA or a viral vector, such as an adeno-associated virus vector, an adenovirus vector, a retroviral vector, etc. In some embodiments, the viral vectors of the application are administered as virus particles, and in others they are administered as plasmids (e.g. as “naked” DNA).

Other methods for the delivery of the nucleotide sequences include those which are already known in the art. These would include the delivery of the nucleotide sequences, such as but not limited to DNA, RNA, siRNA, mRNA, oligonucleotides, or variants thereof, encoding the dual cytokine fusion protein by a cell penetrating peptide, a hydrophobic moiety, an electrostatic complex, a liposome, a ligand, a liposomal nanoparticle, a lipoprotein (preferably HDL or LDL), a folate targeted liposome, an antibody (such as Folate receptor, transferrin receptor), a targeting peptide, or by an aptamer. The nucleotide sequences encoding dual cytokine fusion protein may be delivered to a subject by direct injection, infusion, patches, bandages, mist or aerosol, or by thin film delivery. The nucleotide (or the protein) may be directed to any region that is desired for targeted delivery of a cytokine stimulus. These would include, for example, the lung, the GI tract, the skin, liver, brain though intracranial injection, deep seated metastatic tumor lesions via ultrasound guided injections.

In another aspect, the present disclosure relates to methods of preparing and purifying the dual cytokine fusion protein. For example, nucleic acid sequences that encode the dual cytokine fusion protein described herein may be used to recombinantly produce the fusion proteins. For example, using conventional molecular biology and protein expression techniques, the dual cytokine fusion protein described herein may be expressed and purified from mammalian cell systems. These systems include well known eukaryotic cell expression vector systems and host cells. A variety of suitable expression vectors may be used and are well known to a person skilled in the art, which can be used for expression and introduction of the dual cytokine fusion proteins. These vectors include, for example, pUC-type vectors, pBR-type vectors, pBI-type vectors, pGA-type, pBinI9, pBI121, pGreen series, pCAMBRIA series, pPZP series, pPCV001, pGA482, pCLD04541, pBIBAC series, pYLTAC series, pSB11, pSB1, pGPTV series, and viral vectors and the like can be used. Well known host cell systems include but not limited to expression in CHO cells.

The expression vectors harboring the dual cytokine fusion protein may also include other vector componentry required for vector functionality. For example, the vector may include signal sequences, tag sequences, protease identification sequences, selection markers and other sequences regulatory sequences, such as promoters, required for proper replication and expression of the dual cytokine fusion protein. The particular promoters utilized in the vector are not particularly limited as long as they can drive the expression of the dual cytokine fusion protein in a variety of host cell types. Likewise, the type of Tag promoters are not be limited as long as the Tag sequence makes for simpler or easier purification of expressed dual cytokine fusion protein easier. These might include, for example, 6-histidine, GST, MBP, HAT, HN, S, TF, Trx, Nus, biotin, FLAG, myc, RCFP, GFP and the like can be used. Protease recognition sequences are not particularly limited, for instance, recognition sequences such as Factor Xa, Thrombin, HRV, 3C protease can be used. Selected markers are not particularly limited as long as these can detect transformed rice plant cells, for example, neomycin-resistant genes, kanamycin-resistant genes, hygromycin-resistant genes and the like can be used.

The dual cytokine fusion protein described above may also include additional amino acid sequences that aid in the recovery or purification of the fusion proteins during the manufacturing process. These may include various sequence modifications or affinity tags, such as but not limited to protein A, albumin-binding protein, alkaline phosphatase, FLAG epitope, galactose-binding protein, histidine tags, and any other tags that are well known in the art. See, e.g., Kimple et al (Curr. Protoc. Protein Sci., 2013, 73: Unit 9.9, Table 9.91, incorporated by reference in its entirety). In one aspect, the affinity tag is an histidine tag having an amino acid sequence of HHHHHH (SEQ ID No.: 42). The histidine tag may be removed or left intact from the final product. In another embodiment, the affinity tag is a protein A modification that is incorporated into the fusion protein (e.g., into the VH region of the fusion proteins described herein). A person of skill in the art will understand that any dual cytokine fusion protein sequence described herein can be modified to incorporate a protein A modification by inserting amino acid point substitutions within the antibody framework regions as described in the art.

In another aspect, the protein and nucleic acid molecules encoding dual cytokine fusion protein may be formulated as a pharmaceutical composition comprising a therapeutically effective amount of the dual cytokine fusion protein and a pharmaceutical carrier and/or pharmaceutically acceptable excipients. The pharmaceutical composition may be formulated with commonly used buffers, excipients, preservatives, stabilizers. The pharmaceutical compositions comprising the dual cytokine fusion protein is mixed with a pharmaceutically acceptable carrier or excipient. Various pharmaceutical carriers are known in the art and may be used in the pharmaceutical composition. For example, the carrier can be any compatible, non-toxic substance suitable for delivering the dual cytokine fusion protein compositions of the application to a patient. Examples of suitable carriers include normal saline, Ringer's solution, dextrose solution, and Hank's solution. Carriers may also include any poloxamers generally known to those of skill in the art, including, but not limited to, those having molecular weights of 2900 (L64), 3400 (P65), 4200 (P84), 4600 (P85), 11,400 (F88), 4950 (P103), 5900 (P104), 6500 (P105), 14,600 (F108), 5750 (P123), and 12,600 (F127). Carriers may also include emulsifiers, including, but not limited to, polysorbate 20, polysorbate 40, polysorbate 60, and polysorbate 80, to name a few. Non-aqueous carriers such as fixed oils and ethyl oleate may also be used. The carrier may also include additives such as substances that enhance isotonicity and chemical stability, e.g., buffers and preservatives, see, e.g., Remington's Pharmaceutical Sciences and U.S. Pharmacopeia: National Formulary, Mack Publishing Company, Easton, Pa. (1984). Formulations of therapeutic and diagnostic agents may be prepared by mixing with physiologically acceptable carriers, excipients, or stabilizers in the form of lyophilized powders, slurries, aqueous solutions or suspensions, for example.

The pharmaceutical composition will be formulated for administration to a patient in a therapeutically effective amount sufficient to provide the desired therapeutic result. Preferably, such amount has minimal negative side effects. In one embodiment, the amount of dual cytokine fusion protein administered will be sufficient to treat or prevent inflammatory diseases or condition. In another embodiment, the amount of dual cytokine fusion protein administered will be sufficient to treat or prevent immune diseases or disorders. In still another embodiment, the amount of dual cytokine fusion protein administered will be sufficient to treat or prevent cancer. The amount administered may vary from patient to patient and will need to be determined by considering the subject's or patient's disease or condition, the overall health of the patient, method of administration, the severity of side-effects, and the like.

An effective amount for a particular patient may vary depending on factors such as the condition being treated, the overall health of the patient, the method route and dose of administration and the severity of side effects. The appropriate dose administered to a patient is typically determined by a clinician using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

The method for determining the dosing of the presently described dual cytokine fusion protein will be substantially similar to that described in U.S. Pat. No. 10,858,412. Generally, the presently described dual cytokine fusion protein will have a dosing in the range of 0.5 microgram/kilogram to 100 micrograms/kilogram. The dual cytokine fusion protein may be administered daily, three times a week, twice a week, weekly, bimonthly, or monthly. An effective amount of therapeutic will impact the level of inflammation or disease or condition by relieving the symptom. For example, the impact might include a level of impact that is at least 10%; at least 20%; at least about 30%; at least 40%; at least 50%; or more such that the disease or condition is alleviated or fully treated.

Compositions of the application can be administered orally or injected into the body. Formulations for oral use can also include compounds to further protect the dual cytokine fusion protein from proteases in the gastrointestinal tract. Injections are usually intramuscular, subcutaneous, intradermal or intravenous. Alternatively, intra-articular injection or other routes could be used in appropriate circumstances. Parenterally administered dual cytokine fusion protein are preferably formulated in a unit dosage injectable form (solution, suspension, emulsion) in association with a pharmaceutical carrier and/or pharmaceutically acceptable excipients. In other embodiments, compositions of the application may be introduced into a patient's body by implantable or injectable drug delivery system.

Testing the Dual Cytokine Fusion Protein

A plurality of screening assays are known and available to those of skill in the art to test for the desired biological function. In one embodiment, the desired biological function includes, but are not limited to, reduced anti-inflammatory response, reduce T-cell stimulation, enhanced T-cell function, enhanced Kupffer cell functionality and reduced mast cell degranulation.

For example, it is known that IL-10 exposure primes T cells to generate and secrete more IFNγ upon T cell receptor stimulation. Simultaneously, IL-10 exposure prevents the secretion of TNFα, IL-6 and other pro-inflammatory cytokines secreted from monocytes/macrophages in response to LPS. IL-10 also suppresses FoxP3⁺CD4⁺ T_{reg} proliferation. In one embodiment, the dual cytokine fusion protein that maximize monocyte/macrophage suppression but lack T cell effects, including both stimulatory and suppressive responses, will be positively selected. In one embodiment, screening for dual cytokine fusion proteins that possess increased anti-inflammatory effects will be positively selected for the treatment of autoimmune, anti-inflammatory disease or both. In another embodiments, dual cytokine fusion proteins that enhance Kupffer cell scavenging and lack T_{reg} suppression will also be selected to develop for treatment of Non-alcoholic Steatotic Hepatitis (NASH) and/or Non-alcoholic Fatty Liver Disease (NAFLD). In yet another embodiment, dual cytokine fusion proteins that maximize T cell biology, including both stimulatory and suppressive responses, and also possesses enhanced Kupffer cell scavenging, will be selected to develop for the treatment of cancer. Various assays and methods of screening the dual cytokine fusion proteins are previously described in co-pending U.S. Pat. No. 10,858,412, which is incorporated by reference in its entirety. See, U.S. application Ser. No. 16/811,718 Specification at pages 39-42.

Methods of Treating and/or Preventing Using the Dual Cytokine

In other aspects, the present disclosure relates to methods of treating and/or preventing malignant diseases or conditions or cancer comprising administering to a subject in need thereof a therapeutically effective amount of the dual cytokine fusion protein comprising a first multi-subunit cytokine (e.g., IL-12 or IL-27) and a second cytokine (such as IFNalpha, IL28, IL29, and IL10). In a preferred embodiment, the dual cytokine fusion protein comprises IL-12 or IL-27 and IL-10 or variants thereof, IFN-alpha, IL28, or IL-29 and variants thereof as the second cytokine. In other embodiments, the 6 CDR regions of the human anti-ebola scFv are substituted with 6 CDRs from an anti-Her2/Neu; an anti-PDGFR; anti-VEGFR1 and anti-VEGFR2, an anti-FGFR; an anti-CD19, an anti-CD20, an anti-CD22, an anti-BCMA, an anti-PSA, an anti-PSMA, an anti-HER3; an anti-EGFR, an anti-CEA, or an anti-GPC3. Preferably the 6 CDRs are obtained from anti-EGFR, or anti-HER2. In another preferred embodiment, the second cytokine is an IL-28, IL-29, IL-10, or IFN-alpha, wherein the IL-10 is either DV07 or DV06.

In still other aspects, the present disclosure relates to methods of treating and/or preventing inflammatory diseases or conditions comprising administering to a subject in need thereof a therapeutically effective amount of the dual cytokine fusion protein. In a preferred embodiment, the inflammatory diseases or disorders include, but are not limited to Crohn's disease, psoriasis, and rheumatoid arthritis (“RA”).

In yet another aspect, the present disclosure relates to methods of treating and/or preventing immune diseases or conditions comprising administering to a subject in need thereof a therapeutically effective amount of the dual cytokine fusion protein.

In other embodiments, the present disclosure also contemplates methods of co-administration or treatment with a second therapeutic agent, e.g., a cytokine, steroid, chemotherapeutic agent, antibiotic, anti-inflammatory agents, or radiation, are well known in the art. These might include combination treatments with other therapeutic agents, such as but not limited to one or more the following: chemotherapeutics, interferon-β, for example, IFNβ-1α and IFN-β-1 β; a protein that simulates myelin basic protein; corticosteroids; IL-1 inhibitors; TNF inhibitors; anti-TNFα antibodies, anti-IL-6 antibodies, IL-1br-Ig fusion, anti-IL-23 antibodies, antibodies to CD40 ligand and CD80; antagonists of IL-12 and IL-23, e.g., antagonists of a p40 subunit of IL-12 and IL-23 (e.g., inhibitory antibodies against the p40 subunit); IL-22 antagonists; small molecule inhibitors, e.g., methotrexate, leflunomide, sirolimus (rapamycin) and analogs thereof, e.g., CCI-779; Cox-2 and cPLA2 inhibitors; NSAIDs; p38 inhibitors; TPL-2; Mk-2; NFkβ inhibitors; RAGE or soluble RAGE; P-selectin or PSGL-1 inhibitors (e.g., small molecule inhibitors, antibodies thereto, e.g., antibodies to P-selectin); estrogen receptor beta (ERB) agonists or ERB-NFkβ antagonists.

Additionally, the combination treatment useful for administration with the dual cytokine fusion protein may include TNF inhibitors include, e.g., chimeric, humanized, effectively human, human or in vitro generated antibodies, or antigen-binding fragments thereof, that bind to TNF; soluble fragments of a TNF receptor, e.g., p55 or p75 human TNF receptor or derivatives thereof, e.g., 75 kdTNFR-IgG (75 kD TNF receptor-IgG fusion protein, ENBREL™), p55 kD TNF receptor-IgG fusion protein; and TNF enzyme antagonists, e.g., TNFα converting enzyme (TACE) inhibitors. Other combination treatment with anti-inflammatory agents/drugs that includes, but not limited to standard non-steroidal anti-inflammatory drugs (NSAIDs) and cyclo-oxygenase-2 inhibitors. NSAID may include aspirin, celecoxib, diclofenac, diflunisal, etodolac, ibuprofen, indomethacin, ketoprofen, ketorolac, nabumetone, naproxen, oxaprozin, piroxicam, salsalate, sulindac, and/or tolmetin. The cyclo-oxygenase-2 inhibitor employed in compositions according to the application could, for example, be celecoxib or rofecoxib.

Additional therapeutic agents that can be co-administered and/or co-formulated with the dual cytokine fusion protein include one or more of: interferon-β, for example, IFN β-1α and IFN β-1β; COPAXONE®; corticosteroids; IL-1 inhibitors; TNF antagonists (e.g., a soluble fragment of a TNF receptor, e.g., p55 or p75 human TNF receptor or derivatives thereof, e.g., 75 kdTNFR-IgG; antibodies to CD40 ligand and CD80; and antagonists of IL-12 and/or IL-23, e.g., antagonists of a p40 subunit of IL-12 and IL-23 (e.g., inhibitory antibodies that bind to the p40 subunit of IL-12 and IL-23); methotrexate, leflunomide, and a sirolimus (rapamycin) or an analog thereof, e.g., CCI-779. Other therapeutic agents may include Imfimzi or Atezolizumb.

For purposes of treating NASH, for example, the dual cytokine fusion protein may be combined with cholesterol lowering agents, such as statins and non-statin drugs. These agents include, but are not limited to simvastatin, atorvastatin, rosuvastatin, lovastatin, pravastatin, gemfibrozil, fluvastatin, cholestyramine, fenofibrate, cholesterol absorption inhibitors, bile acid-binding resins or sequestrants, and/or microsomal triglyceride transfer protein (MTP) inhibitors.

Representative chemotherapeutic agents that may be co-administered with the dual cytokine fusion protein described herein may include for following non-exhaustive list: include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g. paclitaxel (TAXOL® Bristol-Myers Squibb Oncology, Princeton, N.J.) and doxetaxel (Taxotere™, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; Xeloda® Roche, Switzerland; ibandronate; CPT11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and antiandrogens such as flutamide, nilutamide, bicalutamide, leuprolide, and goserelin; and pharmaceutically acceptable salts, acids or derivatives of any of the above.

### EXAMPLES

### Example 1: IL-12 and IL-IFN-Alpha Dual Cytokine Fusion Protein In Vitro Study

To evaluate the in vitro effects of targeting two cytokines to a tumor, a dual cytokine fusion protein comprising IL-12 and IFN-alpha (see FIG. 2 as a representative diagram of the structure, termed “DKα¹²”), are constructed from the following components:
(a) p35 and p40 subunits are terminally fused to a human anti-ebola scFv engrafted with 6 CDRs from any one of anti-EGFR, anti-HER2, anti-VEGFR1, anti-VEGFR1, or anti-CD14 antibody; and
(b) an IFN-alpha (SEQ ID No: 9);
where the IFN-alpha is conjugated or linked in the hinge (or linker) region between the VH and VL of the human anti-ebola scFv engrafted with the 6 CDRs from anti-EGFR, anti-HER2, anti-VEGFR1, anti-VEGFR1, or anti-CD14 antibody.

DKα¹² is generated to evaluate the combined effects of these two cytokines—IL-12 and IFN-alpha—on induction of IFNγ from NK, CD4⁺ and CD8⁺ T cells.

Peripheral blood monocytes, NK, CD4⁺ and CD8⁺ T cells were isolated by magnetic bead positive selection to evaluate the function of DKα¹², and then evaluated in in vitro testing. A series of cellular in vitro assays were used to mimic or model immunological function at different time points in the exposure cycle of a molecule injected subcutaneously in the human body. These assays are described in U.S. Pat. No. 10,858,412 and in U.S. application Ser. No. 17/110,104.

First, the effects of IL-12 alone, IFN-alpha alone, the combined effects of IL-12 and IFN-alpha, and IL-12 incorporated into the single cytokine scFv scaffolding system (see, e.g., FIG. 6, where p35 and p40 subunits substitute for the IL-10 monomers on the terminal ends of the scaffolding system) are compared to the effects of the DKα¹² on monocytes/macrophages. IFN-alpha alone and when incorporated into DKα¹² inhibit LPS mediated induction of IL-10 but induce the release of TRAIL. Treatment with IFN-alpha increases cell surface expression of MHC I and CD80. IL-12 exerts no effect on monocytes.

Second, the effects of IL-12 alone, IFN-alpha alone, the combined effects of IL-12 and IFN-alpha, and IL-12 incorporated into the single cytokine scFv scaffolding system (see, e.g., FIG. 6, where p35 and p40 subunits substitute for the IL-10 monomers on the terminal ends of the scaffolding system) are compared to the effects of DKα¹² on T cells. The T-cell assay has been used to directly elucidate the primary function of IL-10 on CD8⁺ T cells, predominantly the potentiation of IFNγ that is only released upon T cell receptor engagement (Chan, 2015; Mumm J., 2011; Emmerich, 2012). This same assay is also applicable to other cytokines, such as IL-12 and may be used to identify T-cell stimulation. IL-12 alone or when incorporated into DKα¹² induces IFN-alpha secretion in this assay. Treatment of T cells with IFN-alpha induces no secretion of IFN-alpha. Treatment of CD8+ T cells with IFN-alpha leads to appreciable proliferation. Treatment with IFN-alpha and IL-12 in combination or when coupled in the DKα¹² leads to both an increase in T cell proliferation and significantly enhances IFN-alpha secretion.

### Example 2: IL-12 and IFN-Alpha Dual Cytokine Fusion Protein In Vivo Study

Targeting a high affinity IL-10 variant (termed-DV07) via an anti-EGFR scFv (wherein DV07 is fused to a scFv comprising VH and VL obtained from a human anti-ebola ScFv scaffolding comprising 6 engrafted anti-EGFR CDRs; “Degfr:DV07”) into the tumor microenvironment by virtue of generating a stably expressed human EGFR CT26 murine colorectal tumor cell line, was previously shown to exhibit superior anti-tumor function when compared with PEG-rHuIL-10. See, U.S. Pat. No. 10,858,412. Using the same in vivo tumor study, DKα¹² is evaluated and compared to Degfr:DV07 in human EGFR expressing CT26 cell murine tumor cell line.

CT26 (hEGFR⁺) tumor bearing B cell k.o. Balb/C mice, with an average of 100 mm³ tumors were treated with test articles, doses and frequencies as provided shown in Table 5. All test articles were administered subcutaneously in the scruff. All articles were dosed daily for 15 days.

TABLE 5
Test Articles, Doses and Frequencies

| No. | Test article | Dose | | Frequency |
|---|---|---|---|---|
| 1 | Vehicle | 100 | μl (control) | Daily |
| 2 | Degfr:IL-12 | 1 | mg/kg | Daily |
| 3 | DKα¹² | 1 | mg/kg | Daily |
| 4 | DKα¹² | 2 | mg/kg | Daily |
| 5 | DKα¹² | 4 | mg/kg | Daily |

The length and width of tumors were measured every three days by electronic calipers and tumor volume was calculated ((L×W²)/2)). In this example, the terms “Degfr:DV07” is human EGFR targeted DV07; DKα¹²egfr is abbreviated as “DKα¹²” and is human IFN-alpha coupled with IL-12 via the Cetuximab CDR grafted anti-ebola scFv scaffold.

### Methods

In vitro cell culture: CT26^{(hEGFR+)} tumor cells (ATCC) are grown to 70% confluency in complete RPMI, 10% FCS, and 10 ug/mL puromycin. Cells are carried for no more than 3 passages in vitro prior to implantation. Cells are removed from cell culture plate using Accutase (Biolegend) and washed in complete RPMI spinning for 10 minutes at 400 g at 4° C.

Tumor Implantation: Tumor cells are implanted at 1-2×10⁵ cells/mouse in 1004 with or without 50% growth factor reduced Matrigel, 50-100% RPMI subcutaneous in the right flank of B cell knockout or wild-type mice.

### Results

Comparison of Degfr:IL-12 and DKα¹² on tumor growth: Targeting IL-12 to the tumor microenvironment via binding to the EGFR present on the stably transfected tumor cells was previously show to be effective. See U.S. Pat. No. 10,858,412. Using the same tumor system, Degfr:IL-12 versus DKα¹² is compared.

Tumors are measured three times a week (Table 2). Female Balb/C B cell knockout mice with 75 mm³ CT26^{(hEGFR+)} tumors are treated subcutaneously with the test articles and various dosing frequencies For this experiment, the CT26^{(hEGFR+)} cells are implanted at 1-2×10⁵ cells in 0-50% growth factor reduced Matrigel to limit immunization of the mice against tumor antigens.

The anti-tumor effect of Degfr:IL-12 at 1 mg/kg is compared to the same dose of DKα¹² as well as 2 and 4 mg/kg doses.

Safety Assessment of DKα¹²: To test the safety of DKα¹² dosing the weight of tumor bearing mice treated with DKα¹² is evaluated.

Effect of DKα¹² dosing on survival: The survivability of CT26^{(hegfr+)} tumor bearing mice to DKα¹² is assessed.

### Example 3: IL-27 and IL-28 or IL-29 Dual Cytokine Fusion Protein

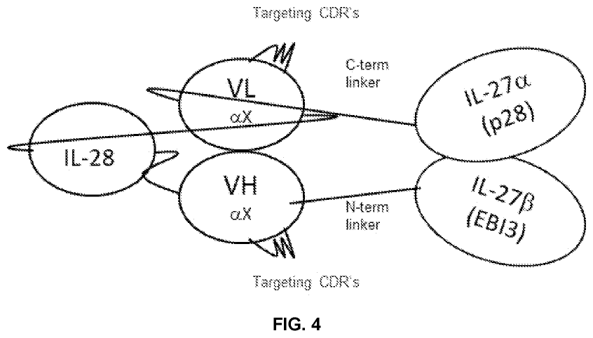

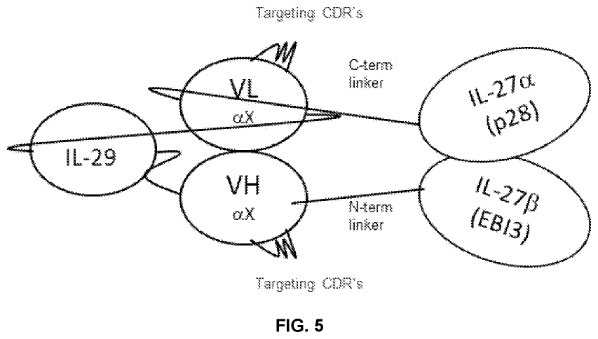

To evaluate the in vitro effects of targeting two cytokines to a tumor, a dual cytokine fusion protein comprising (1) IL-27 and IL-28 (see FIG. 4 as a representative diagram of the structure, termed “DK28²⁷”) and (2) IL-27 and IL-29 (see FIG. 5 as a representative diagram of the structure, termed “DK29²⁷”) are constructed from the following components:

DK28²⁷:
(a) p28 and EBI3 subunits are terminally fused to a human anti-ebola scFv engrafted with 6 CDRs from any one of anti-EGFR, anti-HER2, anti-VEGFR1, anti-VEGFR1, or anti-CD14 antibody; and
(b) an IL-28 (SEQ ID No: 11);
where the IL-28 is conjugated or linked in the hinge (or linker) region between the VH and VL of the human anti-ebola scFv engrafted with the 6 CDRs from anti-EGFR, anti-HER2, anti-VEGFR1, anti-VEGFR1, or anti-CD14 antibody.

DK29²⁷:
(a) p28 and EBI3 subunits are terminally fused to a human anti-ebola scFv engrafted with 6 CDRs from any one of anti-EGFR, anti-HER2, anti-VEGFR1, anti-VEGFR1, or anti-CD14 antibody; and
(b) an IL-29 (SEQ ID No: 16);
where the IL-29 is conjugated or linked in the hinge (or linker) region between the VH and VL of the human anti-ebola scFv engrafted with the 6 CDRs from anti-EGFR, anti-HER2, anti-VEGFR1, anti-VEGFR1, or anti-CD14 antibody.

Both DK28²⁷ and DK29²⁷ are generated to evaluate the combined effects of dual cytokines—IL-27 with IL-28 and IL-28 with IL-29—on induction of IFNγ from NK, CD4⁺ and CD8⁺ T cells. The procedures and assays described above (Example 1) are repeated with these dual cytokines on both monocytes/macrophages, T-cells (CD4+ and CD8+), and NK cells.

### Example 4: IL-27 with IL-28 and IL-27 with IL-29 Dual Cytokine Fusion Protein in Vivo Study

Using the same in vivo tumor study, both DK28²⁷ and DK29²⁷ are evaluated and compared to single targeted cytokine in human EGFR expressing CT26 cell murine tumor cell line. The procedures and assays described above (Example 2) are repeated with these dual cytokines.

### Example 5: IL-12 with IL-10 Dual Cytokine Fusion Protein in an In Vitro Cell Killing Assay Combined with CD19 BiTE

This study was designed to evaluate whether linker lengths have an effect on the cytotoxic function of CD8+ T cells in a molecule that combines IL-12 and IL-10 (DV07) onto an anti-ebola scaffolding engrafted with CDRs that target EGFR (internally designated as DK12¹⁰EGFR) when combined with a CD19 Bispecific T-Cell Engager (CD19 BiTE).

CD8+ T cells were isolated from fresh donor Leukopaks via anti-CD8+ magnetic bead isolation per the manufacturer's suggested protocol (Miltenyi).

The isolated CD8+ T cells were plated at 2×10⁶ cells/well and exposed for 2 days in various concentrations (0 or 200 ng/mL) of DK12¹⁰EGFR in AIMV. Following the 2 days of exposure to the various concentrations of DK12¹⁰EGFR with standard linkers or with DK12¹⁰EGFR having extended linkers on both the IL10 and IL-12 side, the CD8+ T cells were harvested, counted, washed, and finally resuspended in the corresponding concentration of DK12¹⁰EGFR. Concurrently, Raji cells, which constitutively express Green Florescent Protein (GFP), were counted, washed and resuspended in varying concentrations (0 and 0.1 (data not shown), or 1 ng/mL) of CD19 BiTE. The CD8+ cells (effector) and Raji-GFP cells (target) are then combined at a 10:1 effector to target ratio. The mixture of effector and target cells, which are exposed to (1) no treatment, (2) CD19 BiTE alone, (3) DK2¹⁰EGFR alone, or (4) the combination of CD19 BiTE and DK2¹⁰EGFR, were monitored over 2 days using an IncuCyte. Following the 2-day exposure, the percentage of GFP disappearance is measured as an indicator of cytotoxicity.

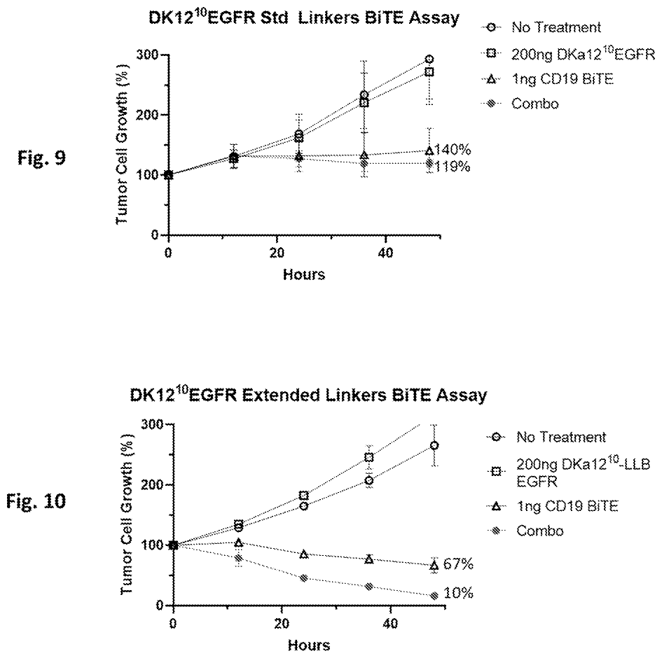

CD19 BiTE, also known as Blinatumomab, is currently the only FDA approved BiTE therapy. We have shown (data not provided) that combining CD19 BiTE with other dual cytokine fusion proteins including IL10 and IL2 (internally termed DK210) enhances CD19 BiTE cytotoxicity. Here we use the same assay system to determine whether combining CD19 BiTE with (1) DK12¹⁰EGFR having standard linkers (e.g., SEQ ID No: 46) would be improved over (2) DK12¹⁰EGFR having extended linkers (e.g., SEQ ID No:44) would drive normal, healthy human donor derived CD8+ T cells to cytolyze target cancer cells. See FIGS. 9 and 10.

The assessment of healthy human donor derived CD8+ T cells to respond to CD19 BiTE with both DK12¹⁰EGFR having standard linkers versus DK12¹⁰EGFR having extended linkers suggests that the extended linkers improve the overall capability of combining the two modalities to enhance CD8+ T cell cytolysis of target Raji cells.

This written description uses examples to disclose aspects of the present disclosure, including the preferred embodiments, and also to enable any person skilled in the art to practice the aspects thereof, including making and using any devices or systems and performing any incorporated methods. The patentable scope of these aspects is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims. Aspects from the various embodiments described, as well as other known equivalents for each such aspect, can be mixed and matched by one of ordinary skill in the art to construct additional embodiments and techniques in accordance with principles of this application.

### REFERENCES

Assier, E. (2004). NK Cells and Polymorphonuclear Neutrophils Are Both Critical for IL-2-Induced Pulmonary Vascular Leak Syndrome. Journal of Immunology.
Athie-Morales, V. (2004). Sustained IL-12 Signaling Is Required for Th1 Development. Journal of Immunology.
Atkins, M. (1997). Phase I evaluation of intravenous recombinant human interleukin 12 in patients with advanced malignancies. Clinical Cancer Research.
Balce, D. R. (2011). Alternative activation of macrophages by IL-4 enhances the proteolytic capacity of their phagosomes through synergistic mechanisms. Blood.
Baluna, R. (1997). Vascular leak syndrome a side effect of immunotherapy. Immunopharmacology.
Bentebibe, S.-E. (2019). A First-in-Human Study and Biomarker Analysis of NKTR-214, a Novel IL2Raf Biased Cytokine, in Patients with Advanced or Metastatic Solid Tumors. Cancer Discovery.
Brunda, M. J. (1993). Antitumor and antimetastatic activity of interleukin 12 against murine tumors. Journal of Experimental Medicine.
Buchbinder, E. I. (2019). Therapy with high-dose Interleukin-2 (HD IL-2) in metastatic melanoma and renal cell carcinoma following PD1 or PDL1 inhibition. Journal of Immunotherapy for Cancer.
Chan, I. H. (2015). The Potentiation of IFNg and Induction of Cytotoxic Proteins by Pegylated IL-10 in Human CD8 T cells. Journal of Interferon and Cytokine Research.
Chen, X. (2018). A novel human IL-2 mutein with minimal systemic toxicity exerts greater antitumor efficacy than wild-type IL-2. Cell Death and Disease.
Chinen, T. (2016). An essential role for IL-2 receptor in regulatory T cell function. Nature Immunology.
Chowdhury, F. Z. (2011). IL-12 selectively programs effector pathways that are stably expressed in human CD8+ effector memory T cells in vivo. Blood.
Coma, G. (2006). Treatment of monocytes with interleukin (IL)-12 plus IL-18 stimulates survival, differentiation and the production of CXC chemokine ligands (CXCL)8, CXCL9 and CXCL10. Clinical and Experimental Immunology.
Davis, I. D. (2009). A Phase I and Pharmacokinetic Study of Subcutaneously-Administered Recombinant Human Interleukin-4 (rhuIL-4) in Patients with Advanced Cancer. Growth Factors.
Emmerich, J. (2012). IL-10 Directly Activates and Expands Tumor-Resident CD8 [symbol] T Cells without De Novo Infiltration from Secondary Lymphoid Organs. Cancer Research, 3570-3581.
Fedorak, R. (2000). Recombinant Human Interleukin 10 in the Treatment of Patients with Mild to Moderately Active Crohn's Disease. Gastroenterology, 1473-1482.
Gessani, S. (2014). Type I Interferons as Regulators of Human Antigen Presenting Cell Functions. Toxins.
Gooch, J. L. (1998). Interleukin 4 Inhibits Growth and Induces Apoptosis in Human Breast Cancer Cells. Cancer Research.
Greve, J. M. (2000). U.S. Pat. No. 6,028,176.
Groux, H. (1998). Inhibitory and Stimulatory Effects of IL-10 on Human CD8+ T cells. The Journal of Immunology.
Guan, H. (2007). Blockade of Hyaluronan Inhibits IL-2 Induced Vascular Leak Syndrome and Maintains Effectiveness of IL-2 Treatment in Metastatic Melanoma. Journal of Immunology.
Hart, P. H. (1989). Potential antiinflammatory effects of interleukin 4: Suppression of human monocyte tumor necrosis factor ca, interleukin 1, and prostaglandin E2. PNAS.
Hart, P. H. (1991). IL-4 suppresses IL-1, TNF-a and PGE2 production by human peritoneal macrophages. Immunology.
Henry, C. J. (2008). IL-12 Produced by Dendritic Cells Augments CD8+ T cell Activation through the Production of the Chemokines CCL1 and CCL17. Journal of Immunology.
Herpen, C. M. (2004). Intratumoral Administration of Recombinant Human Interleukin 12 in Head and Neck Squamous Cell Carcinoma Patients Elicits a T-Helper 1 Profile in the Locoregional Lymph Nodes. Clinical Cancer Research.
Hiroishi, K. (2000). IFN-α-Expressing Tumor Cells Enhance Generation and Promote Survival of Tumor-Specific CTLs. Journal of Immunology.
How, J. (2020). Use of Interferon Alfa in the Treatment of Myeloproliferative Neoplasms: Perspectives and Review of the Literature. Cancers.
Jiang, T. (2016). Role of IL-2 in cancer immunotherapy. Oncoimmunology.
Kirchner, G. I. (1998). Pharmacokinetics of human Interleukin-2 in advanced renal cell carcinoma patients following subcutaneous application. British Journal Clinical Pharmacology.
Kolumam, G. A. (2005). Type I interferons act directly on CD8 T cells to allow clonal expansion and memory formation in response to viral infection. Journal of Experimental Medicine.
Lee, H. L. (2016). Tumor growth suppressive effect of IL-4 through p21-mediated activation of STAT6 in IL-4Rα overexpressed melanoma models. Oncotarget.
Li, R. (2013). Expression of recombinant human IL-4 in Pichia pastoris and relationship between its glycosylation and biological function. Protein Expression and Purification.
Li, S. (2005). Administration Route- and Immune Cell Activation-Dependent Tumor Eradication by IL12 Electrotransfer. Molecular Therapy.
Lu, C. (2019). Type I interferon suppresses tumor growth through activating the STAT3-granzyme B pathway in tumor-infiltrating cytotoxic T lymphocytes. Journal of Immunotherapy of Cancer.
Lyrdal, D. (2009). Metastatic renal cell carcinoma treated with PEG-interferon alfa 2b. Acta Oncology.
Malefyt, R. d. (1991). Interleukin 10 inhibits cytokine synthesis by human monocytes: An autoregulatory role of IL-10 produced by monocytes. JEM.
Malefyt, R. d. (1991). Interleukin 10 Inhibits Cytokine Synthesis by Human Monocytes An
Autoregulatory Role of IL-10 Produced by Monocytes. Journal of Experimental Medicine, 1209-1220.
Martinović, K. M. (2015). Favorable in vitro effects of combined IL-12 and IL-18 treatment on NK cell cytotoxicity and CD25 receptor expression in metastatic melanoma patients. Journal of Translational Medicine.
McGuirk, P. (2000). A Regulatory Role for Interleukin 4 in Differential Inflammatory Responses in the Lung following Infection of Mice Primed with Th1- or Th2-Inducing Pertussis Vaccines. Infection and Immunity.
Medrano, R. F. (2017). Immunomodulatory and antitumor effects of type I interferons and their application in cancer therapy. Oncotarget.
Moore, K. W. (2001). Interleukin 10 and the Interleukin 10 Receptor. Annual Reviews Immunology.
Mossman, T. (1989). THI AND TH2 CELLS: Different Patterns of Lymphokine Secretion Lead to Different Functional Properties. Annual Reviews Immunology.
Mumm, J. (2011). IL-10 induces IFNg-Mediated Tumor Immunity. Cancer Cell.
Mumm, J. B. (2011). IL-10 Elicits IFNg-Dependent Tumor Immune Surveillance. Cancer Cell.
Naing, A. (2016). Safety, Antitumor Activity, and Immune Activation of Pegylated Recombinant Human Interleukin-10 (AM0010) in Patients With Advanced Solid Tumors. Journal of Clinical Oncology.
Naing, A. (2018). PEGylated IL-10 (Pegilodecakin) Induces Systemic Immune Activation, CD8+ T cell Invigoration and Polyclonal T cell Expansion in Cancer Patients. Cancer Cell.
Nemunaitis, J. (2005). Vaccines in Cancer: GVAX® a GM-CSF Gene Vaccine. Vaccine.
Padovan, E. (2002). IFN-α2a induces IP-10/CXCL10 and MIG/CXCL9 production in monocyte-derived dendritic cells and enhances their capacity to attract and stimulate CD8 effector T cells. Journal of Leukocyte Biology.
Parihar, R. (2002). IL-12 enhances the natural killer cell cytokine response to Ab-coated tumor cells. Journal of Clinical Investigation.
Ryan, J. J. (1997). Interleukin-4 and its receptor: Essential mediators of the allergic response. The Journal of Allergy and Clinical Immunology.
Sabel, M. S. (2004). Annals of Surgical Oncology.
Schreiber, S. (2000). Safety and Efficacy of Recombinant Human Interleukin 10 in Chronic Active Crohn's Disease. Gastroenterology, 1461-1472.
Scott, M. J. (2006). Interleukin-10 suppresses natural killer cell but not natural killer T cell activation during bacterial infection. Cytokine.
Simmons, D. P. (2012). Type I interferon drives a distinctive dendritic cell maturation phenotype that allows continued class II MHC synthesis and antigen processing. Journal of Immunology.
Sivakumar, P. V. (2013). Comparison of Vascular Leak Syndrome in Mice Treated with IL21 or IL2. Comparative Medicine.
Spigel, D. R. (2020). Randomized phase II study of pembrolizumab (P) alone versus pegilodecakin (PEG) in combination with P as first-line (1L) therapy in patients (pts) with stage IV non-small cell lung cancer (NSCLC) with high PD-L1 expression (CYPRESS 1). ASCO, (p. 9563).
Steinke, J. W. (2001). Th2 cytokines and asthma Interleukin-4: its role in the pathogenesis of asthma, and targeting it for asthma treatment with interleukin-4 receptor antagonists. Respiratory Research.
Sunela, K. L. (2009). A phase-II study of combination of pegylated interferon alfa-2a and capecitabine in locally advanced or metastatic renal cell cancer. Cancer Chemotherapy and Pharmacology.
Vacaflores, A. (2017). Pretreatment of activated human CD8 T cells with IL-12 leads to enhanced TCR-induced signaling and cytokine production. Molecular Immunology
Vacaflores1, A. (2016). Exposure of Human CD4 T Cells to IL-12 Results in Enhanced TCR-Induced Cytokine Production, Altered TCR Signaling, and Increased Oxidative Metabolism. PLOS One.
Varin, A. (2010). Alternative activation of macrophages by IL-4 impairs phagocytosis of pathogens but potentiates microbial-induced signalling and cytokine secretion. Blood.
Vidyarthi, A. (2018). TLR-3 Stimulation Skews M2 Macrophages to M1 Through IFN-αβ Signaling and Restricts Tumor Progression. Frontiers In Immunology.
Woodward, E. A. (2012). The anti-inflammatory actions of IL-4 in human monocytes are not mediated by IL-10, RP105 or the kinase activity of RIPK2. Cytokine.
Yoo, J. K. (2002). IL-12 Provides Proliferation and Survival Signals to Murine CD4+ T Cells Through Phosphatidylinositol 3-Kinase/Akt Signaling Pathway. Journal of Immunology.
Zhang, C. (2008). Interleukin-12 improves cytotoxicity of natural killer cells via upregulated expression of NKG2D. Human Immunology.

1. A dual cytokine fusion protein of formula (Ia) or (Ib)
NH₂—(R¹)—(X¹)—(Zₙ)—(X²)—(R²)—COOH (Formula Ia);
NH₂—(R²)—(X¹)—(Zₙ)—(X²)—(R¹)—COOH (Formula Ib)
wherein
“R¹” is an alpha subunit of a first cytokine sequence selected from SEQ ID No: 1 17 or 19 or 5;
“R²” is a beta subunit of a first cytokine sequence selected from SEQ ID No: 3, 18, 20, or 7;
wherein when R¹ is SEQ ID No: 1, 17 or 19, R² is SEQ ID No: 3, 18, or 20 or when R¹ is SEQ ID No: 5, R² is SEQ ID No:7;
“X¹” is a VL or VH region obtained from a first monoclonal antibody;
“X²” is a VH or VL region obtained from the first monoclonal antibody;
wherein when X¹ is a VL, X² is a VH or when X¹ is a VH, X² is a VL
“Z” is a cytokine;
“n” is an integer selected from 0-2.

2. The dual cytokine fusion protein according to claim 1, wherein X¹ and X² are obtained from the first monoclonal antibody specific for epidermal growth factor receptor (EGFR); CD14; CD52; various immune check point targets, such as but not limited to PD-L1, PD-1, TIM3, BTLA, LAG3 or CTLA4; CD20; CD47; GD-2; VEGFR1, VEGFR2; HER2; PDGFR; EpCAM; ICAM (ICAM-1, -2, -3, -4, -5), VCAM, FAPα; 5T4; Trop2; EDB-FN; TGFβ Trap; MAdCAM, β7 integrin subunit; α4β7 integrin; α4 integrin SR-A1; SR-A3; SR-A4; SR-A5; SR-A6; SR-B; dSR-C1; SR-D1; SR-E1; SR-F1; SR-F2; SR-G; SR-H1; SR-H2; SR-I1; SR-J1; HIV, or Ebola.

3. The dual cytokine fusion protein according to claim 1, wherein the VL and VH are obtained from the first monoclonal antibody that is an anti-HIV or anti-Ebola antibody.

4. The dual cytokine fusion protein according to claim 3, wherein the VL and VH from the anti-HIV or anti-Ebola monoclonal antibody include 6 CDRs that are engrafted (substituted) with 6 CDRs from a second antibody.

5. The dual cytokine fusion protein according to claim 4, wherein the second antibody is a monoclonal antibody selected from epidermal growth factor receptor (EGFR); CD14; CD52; various immune check point targets, such as but not limited to PD-L1, PD-1, TIM3, BTLA, LAG3 or CTLA4; CD20; CD47; GD-2; VEGFR1; VEGFR2; HER2; PDGFR; EpCAM; ICAM (ICAM-1, -2, -3, -4, -5), VCAM, FAPα; 5T4; Trop2; EDB-FN; TGFβ Trap; MAdCAM, β7 integrin subunit; α4β7 integrin; α4 integrin SR-A1; SR-A3; SR-A4; SR-A5; SR-A6; SR-B; dSR-C1; SR-D1; SR-E1; SR-F1; SR-F2; SR-G; SR-H1; SR-H2; SR-I1; or SR-J1.

6. The dual cytokine fusion protein according to claim 4, wherein the 6 engrafted CDRs from the second monoclonal antibody comprise 6 CDRs from an anti-EGFR antibody, an anti-HER2 antibody, an anti-VEGFR1 antibody, or an anti-VEGFR2 antibody wherein the 6 CDRs comprise CDR 1-3 from the VL and CDR 1-3 from VH.

7. The dual cytokine fusion protein according to claim 1, wherein Z is a cytokine selected from IL-6, IL-4, IL-1, IL-2, IL-3, IL-5, IL-7, IL-8, IL-9, IL-15, IL-21, IL-26, IL-28, IL-29, GM-CSF, G-CSF, interferons-α, -β, -γ, TGF-β, or tumor necrosis factors-α, -β, basic FGF, EGF, PDGF, IL-4, IL-11, or IL-13.

8. The dual cytokine fusion protein according to claim 1, wherein Z is an interferon-α, IL-28, or IL-29.

9. The dual cytokine fusion protein according to claim 1, wherein Z is an integer of 1.

10. The dual cytokine fusion protein according to claim 1, further comprising linkers between each of R¹, X¹, Z, X², and R².

11. A dual cytokine fusion protein comprising IL-12, wherein the fusion protein is Formula (IIa) or (IIb)
NH₂-(p35)-(L)-(X¹)-(L)-(Zₙ)-(L)-(X²)-(L)-(p40)-COOH (Formula IIa);
NH₂-(p40)-(L)-(X¹)-(L)-(Zₙ)-(L)-(X²)-(L)-(p35)-COOH (Formula IIb);
wherein
“p35” is a sequence of SEQ ID No: 1, 17, 19;
“p40” is a sequence of SEQ ID No: 3, 18, 20;
“L” is a linker;
“X¹” is a VL or VH region obtained from a first monoclonal antibody;
“X²” is a VH or VL region obtained from the first monoclonal antibody;
wherein when X¹ is a VL, X² is a VH or when X¹ is a VH, X² is a VL;
“Z” is a cytokine selected from IL-6, IL-4, IL-1, IL-2, IL-3, IL-5, IL-7, IL-8, IL-9, IL-15, IL-21, IL-26, IL-27, IL-28, IL-29, GM-CSF, G-CSF, interferons-α, -γ, TGF-β, or tumor necrosis factors-α, -β, basic FGF, EGF, PDGF, IL-4, IL-11, or IL-13;
“n” is an integer selected from 0-2.

12. The IL-12 fusion protein according to claim 11, wherein the VL and VH are obtained from the first antibody specific for epidermal growth factor receptor (EGFR); CD14; CD52; various immune check point targets, such as but not limited to PD-L1, PD-1, TIM3, BTLA, LAG3 or CTLA4; CD20; CD47; GD-2; VEGFR1; VEGFR2; HER2; PDGFR; EpCAM; ICAM (ICAM-1, -2, -3, -4, -5), VCAM, FAPα; 5T4; Trop2; EDB-FN; TGFβ Trap; MAdCAM, β7 integrin subunit; α4β7 integrin; α4 integrin SR-A1; SR-A3; SR-A4; SR-A5; SR-A6; SR-B; dSR-C1; SR-D1; SR-E1; SR-F1; SR-F2; SR-G; SR-H1; SR-H2; SR-I1; SR-J1; HIV, or Ebola.

13. The IL-12 fusion protein according to claim 12, wherein the VL and VH are obtained from the first antibody specific for HIV or Ebola.

14. The IL-12 fusion protein according to claim 13, wherein the VL and VH from the anti-HIV or anti-Ebola include 6 CDRs that are grafted (substituted) with 6 CDRs from a second antibody.

15. The IL-12 fusion protein according to claim 14, wherein the second antibody is an antibody selected from epidermal growth factor receptor (EGFR); CD14; CD52; various immune check point targets, such as but not limited to PD-L1, PD-1, TIM3, BTLA, LAG3 or CTLA4; CD20; CD47; GD-2; VEGFR1; VEGFR2; HER2; PDGFR; EpCAM; ICAM (ICAM-1, -2, -3, -4, -5), VCAM, FAPα; 5T4; Trop2; EDB-FN; TGFβ Trap; MAdCAM, β7 integrin subunit; α4β7 integrin; α4 integrin SR-A1; SR-A3; SR-A4; SR-A5; SR-A6; SR-B; dSR-C1; SR-D1; SR-E1; SR-F1; SR-F2; SR-G; SR-H1; SR-H2; SR-I1; or SR-J1.

16. The IL-12 fusion protein according to claim 14, wherein the 6 engrafted CDRs from the second antibody comprise 6 CDRs from an antibody selected from an anti-EGFR antibody, an anti-VEGFR1 or VEGFR2 antibody, an anti-HER2 antibody, or an anti-CD14 antibody, wherein the 6 CDRs comprise CDRs 1-3 from the VL and CDRs 1-3 from VH.

17. A dual cytokine fusion protein comprising IL-27, said fusion protein is Formula (IIIa) or (IIIb)
NH₂-(p28)-(L)-(X¹)-(L)-(Zₙ)-(L)-(X²)-(L)-(EBI3)-COOH (Formula IIIa);
NH₂-(EBI3)-(L)-(X¹)-(L)-(Zₙ)-(L)-(X²)-(L)-(p28)-COOH (Formula IIIb);
wherein
“p28” is a sequence of SEQ ID No: 5;
“EBI3” is a sequence of SEQ ID No: 7;
“L” is a linker;
“X¹” is a VL or VH region obtained from a first monoclonal antibody;
“X²” is a VH or VL region obtained from the first monoclonal antibody;
wherein when X¹ is a VL, X² is a VH or when X¹ is a VH, X² is a VL;
“Z” is a cytokine selected from IL-6, IL-4, IL-1, IL-2, IL-3, IL-5, IL-7, IL-8, IL-9, IL-15, IL-21, IL-26, IL-28, IL-29, GM-CSF, G-CSF, interferons-α, -β, -γ, TGF-β, or tumor necrosis factors-α, -β, basic FGF, EGF, PDGF, IL-4, IL-11, or IL-13;
“n” is an integer selected from 0-2.

18. The IL-27 fusion protein according to claim 17, wherein the VL and VH are obtained from the first antibody specific for epidermal growth factor receptor (EGFR); CD14; CD52; various immune check point targets, such as but not limited to PD-L1, PD-1, TIM3, BTLA, LAG3 or CTLA4; CD20; CD47; GD-2; VEGFR1; VEGFR2; HER2; PDGFR; EpCAM; ICAM (ICAM-1, -2, -3, -4, -5), VCAM, FAPα; 5T4; Trop2; EDB-FN; TGFβ Trap; MAdCAM, β7 integrin subunit; α4β7 integrin; α4 integrin SR-A1; SR-A3; SR-A4; SR-A5; SR-A6; SR-B; dSR-C1; SR-D1; SR-E1; SR-F1; SR-F2; SR-G; SR-H1; SR-H2; SR-I1; SR-J1; HIV, or Ebola.

19. The IL-27 fusion protein according to claim 18, wherein the VL and VH are obtained from the first antibody specific for HIV or Ebola.

20. The IL-27 fusion protein according to claim 19, wherein the VL and VH from the anti-HIV or anti-Ebola include 6 CDRs that are grafted (substituted) with 6 CDRs from a second antibody.

21. The IL-27 fusion protein according to claim 20, wherein the second antibody is an antibody selected from epidermal growth factor receptor (EGFR); CD14; CD52; various immune check point targets, such as but not limited to PD-L1, PD-1, TIM3, BTLA, LAG3 or CTLA4; CD20; CD47; GD-2; VEGFR1; VEGFR2; HER2; PDGFR; EpCAM; ICAM (ICAM-1, -2, -3, -4, -5), VCAM, FAPα; 5T4; Trop2; EDB-FN; TGFβ Trap; MAdCAM, β7 integrin subunit; α4β7 integrin; α4 integrin SR-A1; SR-A3; SR-A4; SR-A5; SR-A6; SR-B; dSR-C1; SR-D1; SR-E1; SR-F1; SR-F2; SR-G; SR-H1; SR-H2; SR-I1; or SR-J1.

22. The IL-27 fusion protein according to claim 21, wherein the 6 engrafted CDRs from the second antibody comprise 6 CDRs from an anti-EGFR antibody, an anti-VEGFR1 or VEGFR2 antibody, an anti-HER2 antibody, or an anti-CD14 antibody, wherein the 6 CDRs comprise CDRs 1-3 from the VL and CDRs 1-3 from VH.

23. A method of treating cancer comprising administering to a subject in need thereof, an effective amount of the fusion protein according to claim 1.

24. The method according to claim 23, wherein the fusion protein comprises VL and VH regions from a first antibody selected from anti-HIV or anti-Ebola and wherein the 6 CDR regions of the first antibody are engrafted with 6 CDR regions from second antibody selected from an anti-EGFR antibody, an anti-HER2 antibody, an anti-VEGFR1 antibody, or an anti-VEGFR2 antibody, or anti-CD14.

25. The method according to claim 24, wherein the first antibody is an anti-Ebola antibody and the second antibody is selected from an anti-EGFR antibody, an anti-HER2 antibody, an anti-VEGFR1 antibody, an anti-VEGFR2 antibody, or an anti-CD14 antibody.

26. The method according to claim 25, wherein the first cytokine is an IL-12 or IL-27.

27. The method according to claim 26, wherein the cytokine or “Z” is IL-10.

28. The method according to claim 18, wherein “Z” has a “n” value of 1.

29. The method according to claim 15, wherein the fusion protein is administered at 0.01 ng/ml to 100 ng/ml.

30. The method according to claim 15, wherein the fusion protein is administered at 0.01 ng/ml to 10 ng/ml.


SEQUENCE LISTING (48 sequences)

SEQ ID NO: 1 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 2 - RNA, 1316 nt, Homo sapiens
  source 1..1316: mol_type = mRNA
ggtcaccgagaagctgatgtagagagagacacagaaggagacagaaagcaagagaccagagtcccgggaaagtcctgccgcgcctcgggacaattataaaaatgtggccccctgggtcagcctcccagccaccgccctcacctgccgcggccacaggtctgcatccagcggctcgccctgtgtccctgcagtgccggctcagcatgtgtccagcgcgcagcctcctccttgtcgctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtccctccaaaccgttgtcatttttataaaactttgaaatgaggaaactttgataggatgtggattaagaactagggagggggaaagaaggatgggactattacatccacatgatacctctgatcaagtatttttgacatttactgtggataaattgtttttaagttttcatgaatgaattgctaagaagggaaaatatccatcctgaaggtgtttttcattcactttaatagaagggcaaatatttataagctatttctgtaccaaagtgtttgtggaaacaaacatgtaagcataacttattttaaaatatttatttatataacttggtaatcatgaaagcatctgagctaacttatatttatttatgttatatttattaaattatttatcaagtgtatttgaaaaatatttttaagtgttctaaaaataaaagtattgaatt

SEQ ID NO: 3 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 4 - RNA, 1399 nt, Homo sapiens
  source 1..1399: mol_type = mRNA
ctgtttcagggccattggactctccgtcctgcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttagatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatccatcaggatcagtccctattatgcaaaat

SEQ ID NO: 5 - protein, 243 aa, Homo sapiens
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 6 - RNA, 732 nt, Homo sapiens
  source 1..732: mol_type = mRNA
atgggccagacggcaggcgaccttggctggcgtctcagcctgttgctgcttcccttgctcctggttcaagctggtgtctggggattcccaaggcccccagggaggccccagctgagcctgcaggagctgcggagggagttcacagtcagcctgcatctcgccaggaagctgctctccgaggttcggggccaggcccaccgctttgcggaatctcacctgccaggagtgaacctgtacctcctgcccctgggagagcagctccctgatgtttccctgaccttccaggcctggcgccgcctctctgacccggagcgtctctgcttcatctccaccacgcttcagcccttccatgccccgctgggagggctggggacccagggccgctggaccaacatggagaggatgcagctgtgggccatgaggctggacctccgcgatctgcagcggcacctccgcttccaggtgctggctgcaggattcaacctcccggaggaggaggaggaggaagaggaggaggaggaggaggagaggaaggggctgctcccaggggcactgggcagcgccttacagggcccggcccaggtgtcctggccccagctcctctccacctaccgcctgctgcactccttggagctcgtcttatctcgggccgtgcgggagttgctgctgctgtccaaggctgggcactcagtctggcccttggggttcccaacattgagcccccagccctga

SEQ ID NO: 7 - protein, 229 aa, Homo sapiens
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 8 - RNA, 1161 nt, Homo sapiens
  source 1..1161: mol_type = mRNA
gaattccgcagccatgaccccgcagcttctcctggcccttgtcctctgggccagctgcccgccctgcagtggaaggaaagggcccccagcagctctgacactgccccgggtgcaatgccgagcctctcggtacccgatcgccgtggattgctcctggaccctgccgcctgctccaaactccaccagccccgtgtccttcattgccacgtacaggctcggcatggctgcccggggccacagctggccctgcctgcagcagacgccaacgtccaccagctgcaccatcacggatgtccagctgttctccatggctccctacgtgctcaatgtcaccgccgtccacccctggggctccagcagcagcttcgtgcctttcataacagagcacatcatcaagcccgaccctccagaaggcgtgcgcctaagccccctcgctgagcgccacgtacaggtgcagtgggagcctcccgggtcctggcccttcccagagatcttctcactgaagtactggatccgttacaagcgtcagggagctgcgcgcttccaccgggtggggcccattgaagccacgtccttcatcctcagggctgtgcggccccgagccaggtactacgtccaagtggcggctcaggacctcacagactacggggaactgagtgactggagtctccccgccactgccacaatgagcctgggcaagtagcaagggcttcccgctgcctccagacagcacctgggtcctcgccaccctaagccccgggacacctgttggagggcggatgggatctgcctagcctgggctggagtccttgctttgctgctgctgagctgccgggcaacctcagatgaccgacttttccctttgagcctcagtttctctagctgagaaatggagatgtactactctctcctttacctttacctttaccacagtgcagggctgactgaactgtcactgtgagatattttttattgtttaattagaaaagaattgttgttgggctgggcgcagtggatcgcacctgtaatcccagtcactgggaagccgacgtgggtgggtagcttgaggccaggagctcgaaaccagtccgggccacacagcaagaccccatctctaaaaaattaatataaatataaaataaaaaaaaaaaaaaggaattc

SEQ ID NO: 9 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 10 - RNA, 961 nt, Homo sapiens
  source 1..961: mol_type = mRNA
tgagcctaaaccttaggctcacccatttcaaccagtctagcagcatctgcaacatctacaatggccttgacctttgctttactggtggccctcctggtgctcagctgcaagtcaagctgctctgtgggctgtgatctgcctcaaacccacagcctgggtagcaggaggaccttgatgctcctggcacagatgaggaaaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatgaaaactggttcaacatggaaatgattttcattaattcgtatgccagctcacctttttatgatctgccatttcaaagactcatgtttctgctatgaccatgacacgatttaaatctttttcaaatgtttttaggagtattaatcaacattgtattcagctcttaaggcactagtcccttacagaggaccatgctgactgatccattatctatttaaatatttttaaaatattatttatttaactatttataaaacaacttatttttgttcatattacgtcatgtgcacctttgcacagtggttaatgtaataaaatatgttctttgtatttggtaa

SEQ ID NO: 11 - protein, 200 aa, Homo sapiens
MKLDMTGDCTPVLVLMAAVLTVTGAVPVARLHGALPDARGCHIAQFKSLSPQELQAFKRAKDALEESLLLKDCRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 12 - RNA, 734 nt, Homo sapiens
  source 1..734: mol_type = mRNA
tgggtgacagcctcagagtgtttcttctgctgacaaagaccagagatcaggaatgaaactagacatgactggggactgcacgccagtgctggtgctgatggccgcagtgctgaccgtgactggagcagttcctgtcgccaggctccacggggctctcccggatgcaaggggctgccacatagcccagttcaagtccctgtctccacaggagctgcaggcctttaagagggccaaagatgccttagaagagtcgcttctgctgaaggactgcaggtgccactcccgcctcttccccaggacctgggacctgaggcagctgcaggtgagggagcgccccatggctttggaggctgagctggccctgacgctgaaggttctggaggccaccgctgacactgacccagccctggtggacgtcttggaccagccccttcacaccctgcaccatatcctctcccagttccgggcctgtatccagcctcagcccacggcagggcccaggacccggggccgcctccaccattggctgtaccggctccaggaggccccaaaaaaggagtcccctggctgcctcgaggcctctgtcaccttcaacctcttccgcctcctcacgcgagacctgaattgtgttgccagtggggacctgtgtgtctgaccctcccaccagtcatgcaacctgagattttatttataaattagccacttgtcttaatttattgccacccagtcgctat

SEQ ID NO: 13 - protein, 196 aa, Homo sapiens
MTGDCMPVLVLMAAVLTVTGAVPVARLRGALPDARGCHIAQFKSLSPQELQAFKRAKDALEESLLLKDCKCRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 14 - RNA, 603 nt, Homo sapiens
  source 1..603: mol_type = mRNA
atgaaactagacatgaccggggactgcatgccagtgctggtgctgatggccgcagtgctgaccgtgactggagcagttcctgtcgccaggctccgcggggctctcccggatgcaaggggctgccacatagcccagttcaagtccctgtctccacaggagctgcaggcctttaagagggccaaagatgccttagaagagtcgcttctgctgaaggactgcaagtgccgctcccgcctcttccccaggacctgggacctgaggcagctgcaggtgagggagcgccccgtggctttggaggctgagctggccctgacgctgaaggttctggaggccaccgctgacactgacccagccctgggggatgtcttggaccagccccttcacaccctgcaccatatcctctcccagctccgggcctgtatccagcctcagcccacggcagggcccaggacccggggccgcctccaccattggctgcaccggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 15 - protein, 200 aa, Homo sapiens
MAAAWTVVLVTLVLGLAVAGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 16 - RNA, 856 nt, Homo sapiens
  source 1..856: mol_type = mRNA
aattaccttttcactttacacacatcatcttggattgcccattttgcgtggctaaaaagcagagccatgccgctggggaagcagttgcgatttagccatggctgcagcttggaccgtggtgctggtgactttggtgctaggcttggccgtggcaggccctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgtgtctgagaacgtcaacccaccctgagtccacctgacaccccacaccttatttatgcgctgagccctactccttccttaatttatttcctctcaccctttatttatgaagctgcagccctgactgagacatagggctgagtttattgttttacttttatacattatgcacaaataaacaacaaggaattgga

SEQ ID NO: 17 - protein, 197 aa, Homo sapiens
  source 1..197: note = Human IL-12A (IL-12 p35)
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 18 - protein, 306 aa, Homo sapiens
  source 1..306: note = Human IL-12B (IL-12 p40)
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 19 - protein, 215 aa, Mus musculus
  source 1..215: note = Murine IL-12A (IL-12 p35)
  SIGNAL 1..22
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 20 - protein, 335 aa, Mus musculus
  source 1..335: note = Murine IL-12B (IL-12 p40)
  SIGNAL 1..22
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 21 - protein, 1090 aa, Synthetic construct
  source 1..1090: note = DEK0005:22AASKEC_3253190_DK1210(EGFR)(DK1210 wt12, DV07 w/ regular linker)
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSQVQLQQWGAGLLKPSETLSLTCAVYGFSLTNYGVHWIRQPPGKGLEWLGVIWSGGNTDYNTPFTSRVAISKDNSKNQVSLRLNSVTAADTAIYYCARALTYYDYEFAYWGKGTTVTVSSGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSEIVLTQSPGTLSLSPGERATLSCRASQSIGTNIHWYQQKPGQAPRLLIKYASESISGFPDRFSGSGSGTDFTLTITRLEPEDFAMYYCQQNNNWPTTFGQGTKLEIKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 22 - protein, 1090 aa, Synthetic construct
  source 1..1090: note = DEK0006: 22ABHVJC_3287815_DK1210(EGFR)_degly
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKQKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKQASISVRAQDRYYSSSWSEWASVPCSGGGGSGGGGSGGGGSQVQLQQWGAGLLKPSETLSLTCAVYGFSLTNYGVHWIRQPPGKGLEWLGVIWSGGNTDYNTPFTSRVAISKDNSKNQVSLRLNSVTAADTAIYYCARALTYYDYEFAYWGKGTTVTVSSGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSEIVLTQSPGTLSLSPGERATLSCRASQSIGTNIHWYQQKPGQAPRLLIKYASESISGFPDRFSGSGSGTDFTLTITRLEPEDFAMYYCQQNNNWPTTFGQGTKLEIKGGGGSGGGGSGGGGSRNLPVATPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 23 - protein, 941 aa, Synthetic construct
  source 1..941: note = DEK0014: DKm1210 (hEGFR) 22ACB5AC_3325629
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKQKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKQASISVRAQDRYYSSSWSEWASVPCSGGGGSGGGGSGGGGSQVQLQQWGAGLLKPSETLSLTCAVYGFSLTNYGVHWIRQPPGKGLEWLGVIWSGGNTDYNTPFTSRVAISKDNSKNQVSLRLNSVTAADTAIYYCARALTYYDYEFAYWGKGTTVTVSSGGGGSGGGGSGGGGSDCDIEGKDGKQYESVLMVSIDQLLDSMKEIGSNCLNNEFNFFKRHICDANKEGMFLFRAARKLRQFLKMQSTGDFDLHLLKVSEGTTILLQCTGQVKGRKPAALGEAQPTKSLEEQKSLKEQKKLNDLCFLKRLLQEIKTCWNKILMGTKEHGGGGSGGGGSGGGGSEIVLTQSPGTLSLSPGERATLSCRASQSIGTNIHWYQQKPGQAPRLLIKYASESISGFPDRFSGSGSGTDFTLTITRLEPEDFAMYYCQQNNNWPTTFGQGTKLEIKGGGGSGGGGSGGGGSRNLPVATPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 24 - protein, 1110 aa, Synthetic construct
  source 1..1110: note = DEK0039: 22ACEUSC_3330050_DKa1210(EGFR)_G4Sx5 "V3" (DK1210 w/ 12 side long linker)
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKQKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKQASISVRAQDRYYSSSWSEWASVPCSGGGGSGGGGSGGGGSGGGGSGGGGSQVQLQQWGAGLLKPSETLSLTCAVYGFSLTNYGVHWIRQPPGKGLEWLGVIWSGGNTDYNTPFTSRVAISKDNSKNQVSLRLNSVTAADTAIYYCARALTYYDYEFAYWGKGTTVTVSSGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSEIVLTQSPGTLSLSPGERATLSCRASQSIGTNIHWYQQKPGQAPRLLIKYASESISGFPDRFSGSGSGTDFTLTITRLEPEDFAMYYCQQNNNWPTTFGQGTKLEIKGGGGSGGGGSGGGGSGGGGSGGGGSRNLPVATPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 25 - protein, 1118 aa, Synthetic construct
  source 1..1118: note = DEK0031: 22ACT5OC_3351531_DKa1210_EGFR_G4Sx5v1
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKQKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKQASISVRAQDRYYSSSWSEWASVPCSGGGGSGGGGSGGGGSGGGGSGGGGSQVQLQQWGAGLLKPSETLSLTCAVYGFSLTNYGVHWIRQPPGKGLEWLGVIWSGGNTDYNTPFTSRVAISKDNSKNQVSLRLNSVTAADTAIYYCARALTYYDYEFAYWGKGTTVTVSSGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSEIVLTQSPGTLSLSPGERATLSCRASQSIGTNIHWYQQKPGQAPRLLIKYASESISGFPDRFSGSGSGTDFTLTITRLEPEDFAMYYCQQNNNWPTTFGQGTKLEIKGGGGSGGGGSGGGGSGGGGSGGGGSRNLPVATPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 26 - protein, 1130 aa, Synthetic construct
  source 1..1130: note = DEK0040: 22ACT5PC_3351532_DKa1210_EGFR_G4Sx5v2 (DK1210 w/ 10 and 12 side long linker)
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKQKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKQASISVRAQDRYYSSSWSEWASVPCSGGGGSGGGGSGGGGSGGGGSGGGGSQVQLQQWGAGLLKPSETLSLTCAVYGFSLTNYGVHWIRQPPGKGLEWLGVIWSGGNTDYNTPFTSRVAISKDNSKNQVSLRLNSVTAADTAIYYCARALTYYDYEFAYWGKGTTVTVSSGGGGSGGGGSGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSGGGGSGGGGSEIVLTQSPGTLSLSPGERATLSCRASQSIGTNIHWYQQKPGQAPRLLIKYASESISGFPDRFSGSGSGTDFTLTITRLEPEDFAMYYCQQNNNWPTTFGQGTKLEIKGGGGSGGGGSGGGGSGGGGSGGGGSRNLPVATPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 27 - protein, 1100 aa, Synthetic construct
  source 1..1100: note = DEK0071: DK1210 EGFR 22ABBLTD_3417493 (DK1210 w/ 12 side 4x4GS linker)
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKQKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKQASISVRAQDRYYSSSWSEWASVPCSGGGGSGGGGSGGGGSGGGGSQVQLQQWGAGLLKPSETLSLTCAVYGFSLTNYGVHWIRQPPGKGLEWLGVIWSGGNTDYNTPFTSRVAISKDNSKNQVSLRLNSVTAADTAIYYCARALTYYDYEFAYWGKGTTVTVSSGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSEIVLTQSPGTLSLSPGERATLSCRASQSIGTNIHWYQQKPGQAPRLLIKYASESISGFPDRFSGSGSGTDFTLTITRLEPEDFAMYYCQQNNNWPTTFGQGTKLEIKGGGGSGGGGSGGGGSGGGGSRNLPVATPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 28 - protein, 1091 aa, Synthetic construct
  source 1..1091: note = DEK0017: DK1210 Her2 22ABRXVC_3302173
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSQVQLQQWGAGLLKPSETLSLTCAVYGFNIKDTYIHWIRQPPGKGLEWLGRIYPTNGYTRYADSVKGRVAISKDNSKNQVSLRLNSVTAADTAIYYCARWGGDGFYAMDYWGKGTTVTVSSGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSEIVLTQSPGTLSLSPGERATLSCRASQDVNTAVAWYQQKPGQAPRLLIKSASFLYSGFPDRFSGSGSGTDFTLTITRLEPEDFAMYYCQQHYTTPPTFGQGTKLEIKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 29 - protein, 1112 aa, Synthetic construct
  source 1..1112: note = DEK0041: DKa1210(CD20) "V3" 22ACEUQC_3330048
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKQKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKQASISVRAQDRYYSSSWSEWASVPCSGGGGSGGGGSGGGGSGGGGSGGGGSQAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSSGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKGGGGSGGGGSGGGGSGGGGSGGGGSRNLPVATPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 30 - protein, 1132 aa, Synthetic construct
  source 1..1132: note = DEK0078: DKa1210 (CD20) G4Sx5
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKQKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKQASISVRAQDRYYSSSWSEWASVPCSGGGGSGGGGSGGGGSGGGGSGGGGSQAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSSGGGGSGGGGSGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSGGGGSGGGGSQIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKGGGGSGGGGSGGGGSGGGGSGGGGSRNLPVATPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 31 - protein, 178 aa, Homo sapiens
  source 1..178: note = Human IL-10 Amino Acid Sequence
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 32 - protein, 1629 aa, Homo sapiens
  source 1..1629: note = Human IL-10 Nucleic Acid Sequence
ACACATCAGGGGCTTGCTCTTGCAAAACCAAACCACAAGACAGACTTGCAAAAGAAGGCATGCACAGCTCAGCACTGCTCTGTTGCCTGGTCCTCCTGACTGGGGTGAGGGCCAGCCCAGGCCAGGGCACCCAGTCTGAGAACAGCTGCACCCACTTCCCAGGCAACCTGCCTAACATGCTTCGAGATCTCCGAGATGCCTTCAGCAGAGTGAAGACTTTCTTTCAAATGAAGGATCAGCTGGACAACTTGTTGTTAAAGGAGTCCTTGCTGGAGGACTTTAAGGGTTACCTGGGTTGCCAAGCCTTGTCTGAGATGATCCAGTTTTACCTGGAGGAGGTGATGCCCCAAGCTGAGAACCAAGACCCAGACATCAAGGCGCATGTGAACTCCCTGGGGGAGAACCTGAAGACCCTCAGGCTGAGGCTACGGCGCTGTCATCGATTTCTTCCCTGTGAAAACAAGAGCAAGGCCGTGGAGCAGGTGAAGAATGCCTTTAATAAGCTCCAAGAGAAAGGCATCTACAAAGCCATGAGTGAGTTTGACATCTTCATCAACTACATAGAAGCCTACATGACAATGAAGATACGAAACTGAGACATCAGGGTGGCGACTCTATAGACTCTAGGACATAAATTAGAGGTCTCCAAAATCGGATCTGGGGCTCTGGGATAGCTGACCCAGCCCCTTGAGAAACCTTATTGTACCTCTCTTATAGAATATTTATTACCTCTGATACCTCAACCCCCATTTCTATTTATTTACTGAGCTTCTCTGTGAACGATTTAGAAAGAAGCCCAATATTATAATTTTTTTCAATATTTATTATTTTCACCTGTTTTTAAGCTGTTTCCATAGGGTGACACACTATGGTATTTGAGTGTTTTAAGATAAATTATAAGTTACATAAGGGAGGAAAAAAAATGTTCTTTGGGGAGCCAACAGAAGCTTCCATTCCAAGCCTGACCACGCTTTCTAGCTGTTGAGCTGTTTTCCCTGACCTCCCTCTAATTTATCTTGTCTCTGGGCTTGGGGCTTCCTAACTGCTACAAATACTCTTAGGAAGAGAAACCAGGGAGCCCCTTTGATGATTAATTCACCTTCCAGTGTCTCGGAGGGATTCCCCTAACCTCATTCCCCAACCACTTCATTCTTGAAAGCTGTGGCCAGCTTGTTATTTATAACAACCTAAATTTGGTTCTAGGCCGGGCGCGGTGGCTCACGCCTGTAATCCCAGCACTTTGGGAGGCTGAGGCGGGTGGATCACTTGAGGTCAGGAGTTCCTAACCAGCCTGGTCAACATGGTGAAACCCCGTCTCTACTAAAAATACAAAAATTAGCCGGGCATGGTGGCGCGCACCTGTAATCCCAGCTACTTGGGAGGCTGAGGCAAGAGAATTGCTTGAACCCAGGAGATGGAAGTTGCAGTGAGCTGATATCATGCCCCTGTACTCCAGCCTGGGTGACAGAGCAAGACTCTGTCTCAAAAAATAAAAATAAAAATAAATTTGGTTCTAATAGAACTCAGTTTTAACTAGAATTTATTCAATTCCTCTGGGAATGTTACATTGTTTGTCTGTCTTCATAGCAGATTTTAATTTTGAATAAATAAATGTATCTTATTCACATC

SEQ ID NO: 33 - protein, 147 aa, Human gammaherpesvirus 4
  source 1..147: note = EBV IL-10 Amino Acid Sequence
TDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 34 - DNA, 632 bp, Human gammaherpesvirus 4
  source 1..632: mol_type = genomic DNA; note = EBV IL-10 Nucleic Acid Sequence
tataaatcacttccctatctcaggtaggcctgcacaccttaggtatggagcgaaggttagtggtcactctgcagtgcctggtgctgctttacctggcacctgagtgtggaggtacagaccaatgtgacaattttccccaaatgttgagggacctaagagatgccttcagtcgtgttaaaacctttttccagacaaaggacgaggtagataaccttttgctcaaggagtctctgctagaggactttaagggctaccttggatgccaggccctgtcagaaatgatccaattctacctggaggaagtcatgccacaggctgaaaaccaggaccctgaagccaaagaccatgtcaattctttgggtgaaaatctaaagaccctacggctccgcctgcgcaggtgccacaggttcctgccgtgtgagaacaagagtaaagctgtggaacagataaaaaatgcctttaacaagctgcaggaaaaaggaatttacaaagccatgagtgaatttgacatttttattaactacatagaagcatacatgacaattaaagccaggtgataattccataccctggaagcaggagatgggtgcatttcaccccaaccccccctttcgactgtcatttacaataaa

SEQ ID NO: 35 - protein, 177 aa, Human betaherpesvirus 5
  source 1..177: note = CMV IL-10 Amino Acid Sequence
MLSVMVSSSLVLIVFFLGASEEAKPAATTTTIKNTKPQCRPEDYASRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 36 - DNA, 693 bp, Human betaherpesvirus 5
  source 1..693: mol_type = genomic DNA; note = CMV IL-10 Nucleic Acid Sequence
atgctgtcggtgatggtctcttcctctctggtcctgatcgtcttttttctaggcgcttccgaggaggcgaagccggcggcgacgacgacgacgataaagaatacaaagccgcagtgtcgtccggaggattacgcgagcagattgcaagatctccgcgtcacctttcatcgagtaaaacctacgttggtaggtcatgtaggtacggtttattgcgacggtctttcttttccgcgtgtcgggtgacgtagttttcctcttgtagcaacgtgaggacgactactccgtgtggctcgacggtacggtggtcaaaggctgttggggatgcagcgtcatggactggttgttgaggcggtatctggagatcgtgttccccgcaggcgaccacgtctatcctggacttaagacggaattgcatagtatgcgctcgacgctagaatccatctacaaagacatgcggcaatgcgtaagtgtctctgtggcggcgctgtccgcgcagaggtaacaacgtgttcatagcacgctgttttacttttgtcgggctcccagcctctgttaggttgcggagataagtccgtgattagtcggctgtctcaggaggcggaaaggaaatcggataacggcacgcggaaaggtctcagcgagttggacacgttgtttagccgtctcgaagagtatctgcactcgagaaagtag

SEQ ID NO: 37 - protein, 178 aa, Mus musculus
  source 1..178: note = Mouse IL-10 Amino Acid Sequence
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 38 - DNA, 1314 bp, Mus musculus
  source 1..1314: mol_type = genomic DNA; note = Mouse IL-10 Nucleic Acid Sequence
ggggggggggatttagagacttgctcttgcactaccaaagccacaaagcagccttgcagaaaagagagctccatcatgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagctaaaacacctgcagtgtgtattgagtctgctggactccaggacctagacagagctctctaaatctgatccagggatcttagctaacggaaacaactccttggaaaacctcgtttgtacctctctccgaaatatttattacctctgatacctcagttcccattctatttattcactgagcttctctgtgaactatttagaaagaagcccaatattataattttacagtatttattatttttaacctgtgtttaagctgtttccattggggacactttatagtatttaaagggagattatattatatgatgggaggggttcttccttgggaagcaattgaagcttctattctaaggctggccacacttgagagctgcagggccctttgctatggtgtcctttcaattgctctcatccctgagttcagagctcctaagagagttgtgaagaaactcatgggtcttgggaagagaaaccagggagatcctttgatgatcattcctgcagcagctcagagggttcccctactgtcatcccccagccgcttcatccctgaaaactgtggccagtttgttatttataaccacctaaaattagttctaatagaactcatttttaactagaagtaatgcaattcctctgggaatggtgtattgtttgtctgcctttgtagcagcatctaattttgaataaatggatcttattcg

SEQ ID NO: 39 - protein, 147 aa, synthetic construct
  source 1..147: note = DV06 Amino Acid Sequence
TDQCDNFPQMLRDLRDAFSRVKTFFQTKDEVDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 40 - DNA, 441 bp, synthetic construct
  source 1..441: mol_type = other DNA; note = DV06 Nucleic Acid Sequence
  misc_feature 93: note = n is a, c, g, or t
accgaccagtgcgacaacttccctcagatgctgcgggacctgagagatgccttctccagagtgaaaacattcttccagaccaaggacgaggtngacaacctgctgctgaaagagtccctgctggaagatttcaagggctacctgggctgtcaggccctgtccgagatgatccagttctacctggaagaagtgatgccccaggccgagaatcaggaccccgagatcaaggaccacgtgaactccctgggcgagaacctgaaaaccctgcggctgagactgcggcggtgccacagatttctgccctgcgagaacaagtccaaggccgtggaacagatcaagaacgccttcaacaagctgcaagagaagggcatctacaaggccatgagcgagttcgacatcttcatcaactacatcgaggcctacatgaccatcaaggccaga

SEQ ID NO: 41 - protein, 147 aa, synthetic construct
  source 1..147: note = DV07 Amino Acid Sequence
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 42 - DNA, 441 bp, synthetic construct
  source 1..441: mol_type = other DNA; note = DV07 Nucleic Acid Sequence
accgaccagtgcgacaacttccctcagatgctgcgggacctgagagatgccttctccagagtgaaaacattcttccagaccaaggacgagctggacaacctgctgctgaaagagtccctgctggaagatttcaagggctacctgggctgtcaggccctgtccgagatgatccagttctacctggaagaagtgatgccccaggccgagaatcaggaccccgagatcaaggaccacgtgaactccctgggcgagaacctgaaaaccctgcggctgagactgcggcggtgccacagatttctgccctgcgagaacaagtccaaggccgtggaacagatcaagaacgccttcaacaagctgcaagagaagggcatctacaaggccatgagcgagttcgacatcttcatcaactacatcgaggcctacatgaccatcaaggccaga

SEQ ID NO: 43 - protein, 7 aa, synthetic construct
  source 1..7: note = Linker dimer
GGGSGGG

SEQ ID NO: 44 - protein, 25 aa, synthetic construct
  source 1..25: note = Linker x5
GGGGSGGGGSGGGGSGGGGSGGGGS

SEQ ID NO: 45 - protein, 20 aa, synthetic construct
  source 1..20: note = Linker x4
GGGGSGGGGSGGGGSGGGGS

SEQ ID NO: 46 - protein, 15 aa, synthetic construct
  source 1..15: note = Linker x3
GGGGSGGGGSGGGGS

SEQ ID NO: 47 - protein, 10 aa, synthetic construct
  source 1..10: note = Linker x2
GGGGSGGGGS

SEQ ID NO: 48 - protein, 17 aa, synthetic construct
  source 1..17: note = Linker
SSGGGGSGGGGSGGGGS